(19) United States
(12) Patent Application Publication
(10) Pub. No.: US 2024/0261249 A1
(43) Pub. Date: Aug. 8, 2024

(54) NOVEL SQUARAMIDE DERIVATIVE AND USE THEREOF

(71) Applicants: PUSAN NATIONAL UNIVERSITY INDUSTRY-UNIVERSITY COOPERATION FOUNDATION, Busan (KR); KOSIN UNIVERSITY INDUSTRY-ACADEMY COOPERATION, Busan (KR)

(72) Inventors: Hwayoung YUN, Busan (KR); Jee-Yeong JEONG, Busan (KR); Jinsook KWAK, Busan (KR); Min Jung KIM, Busan (KR); Soyeong KIM, Busan (KR)

(73) Assignees: PUSAN NATIONAL UNIVERSITY INDUSTRY-UNIVERSITY COOPERATION FOUNDATION, Busan (KR); KOSIN UNIVERSITY INDUSTRY-ACADEMY COOPERATION, Busan (KR)

(21) Appl. No.: 18/435,962

(22) Filed: Feb. 7, 2024

(30) Foreign Application Priority Data
Jun. 1, 2021 (KR) 10-2021-0070727

(51) Int. Cl. A61K 31/277; A61K 31/145; A61P 35/00; C07C 255/58; C07C 317/22

(52) U.S. Cl. CPC A61K 31/277; A61K 31/145; A61P 35/00; C07C 255/58; C07C 317/22; C07C 2601/04; C07C 2601/14

(57) ABSTRACT

The present invention relates to a novel squaramide derivative and a use thereof and provides a novel derivative through the strategy of replacing a compound having a urea core with the bioisolate squaramide, whereby the derivative exhibits an anticancer activity through eIF2α phosphorylation efficacy, wherein squaramide has a characteristic structure retaining a double bond linked to the carbonyl groups and the squaramide structure can be prepared by mediating a precursor bearing an amine group to the squarate ring, which is a squaric ring, through a conjugate addition reaction.

### CROSS REFERENCE TO RELATED APPLICATIONS

This application is a Continuation of U.S. patent application Ser. No. 18/565,530 filed on Nov. 30, 2023, which is a National Stage patent application of PCT International Patent Application No. PCT/KR2022/007722 filed on May 31, 2022, which claims priority to Korean Patent Application No. 10-2021-0070727 filed on Jun. 1, 2021 which are all hereby incorporated by reference in their entirety.

### BACKGROUND OF THE INVENTION

The present disclosure relates to a novel squaramide derivative and a use thereof.

Encountering with various stressful situations, cells activate integrated stress response pathways to regulate synthesis of proteins to cope with changes in the cells. The integrated stress response in eukaryotes leads to phosphorylation of eukaryotic translation initiation factor 2α (eIF2α), which is mediated by activation of four types of stress-sensing eIF2α kinases (HRI, PERK, GCN2, and PKR). When phosphorylation of eIF2α takes place, overall protein synthesis is inhibited, and paradoxically, the expression of ATF4, a factor coping with stress, increases, thereby promoting the recovery of cells to their normal state eventually. Since eIF2α is overexpressed during the carcinogenesis process of cells and regulates the synthesis of intracellular proteins through downstream signaling, the phosphorylation of eIF2α may be considered as a target of cancer cell-specific metabolic regulation. Recent studies have reported that it may induce anticancer effects by promoting the phosphorylation of eIF2α, and it has been revealed that the compound with a parent nucleus of urea developed by the Halperin research team has excellent eIF2α phosphorylation efficacy and anticancer activity. However, no candidate with a low molecular weight has been reported, that entered the clinical trial stage so far while exhibiting eIF2α phosphorylation efficacy. Therefore, there is a sufficient need for further research, and it is expected that it will be possible to develop an anticancer drug using this mechanism. Currently, it is best known that metabolic anticancer drugs have good efficacy in leukemia, and studies have also reported that they take effects on rare cancers such as breast cancer, brain tumors, and sarcomas. If a metabolic anticancer drug is developed through regulation of eIF2α phosphorylation, it may be used for treatment of rare cancers associated with abnormal expression of HRI, PERK, GCN2, and PKR, which are the upstream signaling factors of eIF2α, in addition to the aforementioned cancers.

### SUMMARY OF THE INVENTION

An object of the present disclosure is to provide a novel squaramide derivative compound.

Another object of the present disclosure is to provide a pharmaceutical composition for preventing or treating cancer, including the novel squaramide derivative compound as an active ingredient.

Another object of the present disclosure is to provide a health functional food composition for preventing or ameliorating cancer, including the novel squaramide derivative compound as an active ingredient.

### Technical Solutions

To achieve the above objects, the present disclosure provides a compound selected from a squaramide derivative compound represented by the following Chemical Formula 1, pharmaceutically acceptable salt thereof, solvent thereof, or stereoisomer thereof.

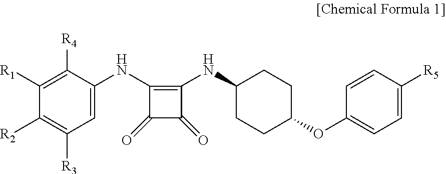

In the Chemical Formula 1,
R₁, R₂, and R₄ may be the same or different respectively and selected from the group consisting of hydrogen (H), hydroxy (OH), halogen, trifluoromethyl (CF₃), (C1˜C4)alkyl, (C1˜C4)alkoxy, and acetate (COOMe),
R₃ is selected from the group consisting of hydrogen (H) and cyano (CN), and
R₅ is selected from the group consisting of trifluoromethyl group (CF₃), cyano (CN), nitrite (NO₂), (C1˜C4)alkylsulfonyl (SO₂Me), and aminosulfonyl (SO₂NH₂).

In addition, the present disclosure provides a pharmaceutical composition for preventing or treating cancer, including the compound as an active ingredient.

In addition, the present disclosure provides a pharmaceutical composition for metabolic anticancer drugs, including the compound as an active ingredient.

In addition, the present disclosure provides a method of inhibiting cancer cell metabolism, including administering the compound.

In addition, the present disclosure provides a health functional food composition for preventing or ameliorating cancer, including the compound as an active ingredient.

### Advantageous Effects

The present disclosure relates to a novel squaramide derivative and a use thereof, and provides a novel derivative that exhibits anticancer activity through eIF2α phosphorylation efficacy by a strategy to substitute a compound having a parent nucleus of urea with squaramide, a bioisostere. Squaramide includes a specific structure that has a double bond linked to a carbonyl group. A squaramide structure may be prepared through mediation via a conjugate addition reaction using a precursor having an amine group in a squarate ring of a square ring. The present disclosure provides a derivative in which a phenoxycyclohexyl structure is introduced in the opposite nitrogen by utilizing a core intermediate that is efficiently constructed by introducing an aniline ring having various substituents into squaramide. The synthesized novel derivative shows an eIF2α phosphorylation effect and cancer cell proliferation inhibitory activity in vitro and thus is applicable as a metabolic anticancer drug.

### BRIEF DESCRIPTION OF DRAWINGS

FIG. 1 shows a designing strategy for N,N′-squaramide derivatives.

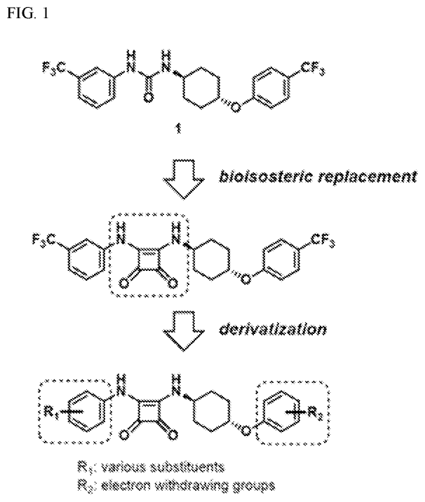

FIG. 2 shows a synthesis process for intermediates 7a-r of squaramide derivatives.

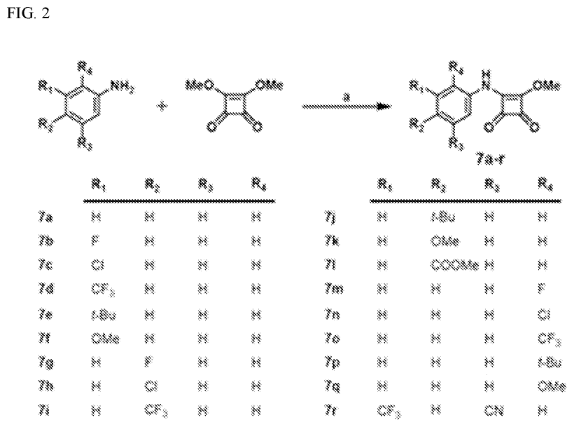

FIG. 3 shows a synthesis process of squaramide derivatives 8-86.

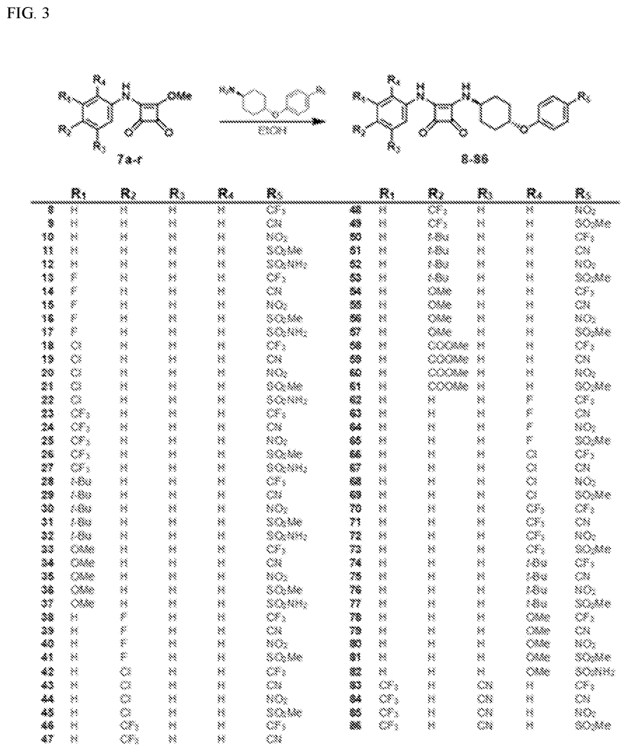

### DETAILED DESCRIPTION OF THE INVENTION

Hereinafter, the present disclosure will be described in more detail.

An important step in initiation of protein synthesis is formation of a complex consisting of eIF2, GTP, and Met-tRNAi. This complex plays an important role in performing normal cellular functions and is attracting attention as a target for the development of new therapeutics as it is known to cause various diseases with an abnormality in its regulation, such that the inventors completed the present disclosure by efficiently constructing a key intermediate in which an aniline ring having various substituents is introduced into squaramide, synthesizing a derivative in which a phenoxycyclohexyl structure is introduced into the opposite nitrogen by utilizing the same, and determining that the new derivative thus synthesized shows the eIF2α phosphorylation effect and cancer cell proliferation inhibitory activity in vitro.

The present disclosure provides a compound selected from a squaramide derivative compound represented by the following Chemical Formula 1, pharmaceutically acceptable salt thereof, solvent thereof, or stereoisomer thereof.

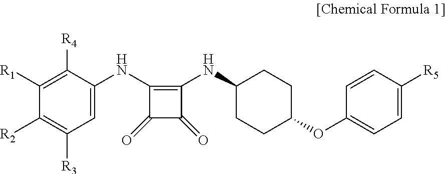

In the Chemical Formula 1,
R₁, R₂, and R₄ may be the same or different respectively and selected from the group consisting of hydrogen (H), hydroxy (OH), halogen, trifluoromethyl (CF₃), (C1˜C4)alkyl, (C1˜C4)alkoxy, and acetate (COOMe),
R₃ may be selected from the group consisting of hydrogen (H) and cyano (CN), and
R₅ may be selected from the group consisting of trifluoromethyl group (CF₃), cyano (CN), nitrite (NO₂), (C1˜C4)alkylsulfonyl (SO₂Me), and aminosulfonyl (SO₂NH₂).

Preferably, in the Chemical Formula 1 of the squaramide derivative compound, R₁ may be selected from the group consisting of hydrogen (H), fluorine (F), chlorine (Cl), (C1˜C2)alkoxy, trifluoromethyl (CF₃), and (C3˜C4)alkyl, R₂ may be selected from the group consisting of hydrogen (H), fluorine (F), chlorine (Cl), trifluoromethyl (CF₃), (C3˜C4)alkyl, (C1˜C2)alkoxy, and acetate (COOMe), R₃ may be selected from the group consisting of hydrogen (H) and cyano (CN), R₄ may be selected from the group consisting of hydrogen (H), fluorine (F), chlorine (Cl), (C1˜C2)alkoxy, trifluoromethyl (CF₃), and (C3˜C4)alkyl, and R₅ may be selected from the group consisting of trifluoromethyl group (CF₃), cyano (CN), nitrite (NO₂), (C1˜C2)sulfonyl (SO₂Me), and aminosulfonyl (SO₂NH₂).

More preferably, in the Chemical Formula 1 of the squaramide derivative compound, R₁ may be selected from the group consisting of hydrogen (H), fluorine (F), chlorine (Cl), methoxy (OMe), trifluoromethyl (CF₃), and t-butyl (t-Bu), R₂ may be selected from the group consisting of hydrogen (H), fluorine (F), chlorine (Cl), trifluoromethyl (CF₃), t-butyl (t-Bu), methoxy (OMe), and acetate (COOMe), R₃ may be selected from the group consisting of hydrogen (H) and cyano (CN), R₄ may be selected from the group consisting of hydrogen (H), fluorine (F), chlorine (Cl), methoxy (OMe), trifluoromethyl (CF₃), and t-butyl (t-Bu), and R₅ may be selected from the group consisting of trifluoromethyl group (CF₃), cyano (CN), nitrite (NO₂), methylsulfonyl (SO₂Me), and aminosulfonyl (SO₂NH₂).

As an embodiment, the squaramide derivative compound may be selected from the group consisting of, but is not limited to, 3-(phenylamino)-4-(((1,4-trans)-4-(4-(trifluoromethyl)phenoxy)cyclohexyl)amino)cyclobut-3-ene-1,2-dione, 4-(((1,4-trans)-4-((3,4-dioxo-2-(phenylamino)cyclobut-1-en-1-yl)amino)cyclohexyl)oxy)benzonitrile, 3-(((1,4-trans)-4-(4-nitrophenoxy)cyclohexyl)amino)-4-(phenylamino)cyclobut-3-ene-1,2-dione, 3-(((1,4-trans)-4-(4-(methylsulfonyl)phenoxy)cyclohexyl)amino)-4-(phenylamino)cyclobut-3-ene-1,2-dione, 4-(((1,4-trans)-4-((3,4-dioxo-2-(phenylamino)cyclobut-1-en-1-yl)amino)cyclohexyl)oxy)benzenesulfonamide, 3-((3-fluorophenyl)amino)-4-(((1,4-trans)-4-(4-(trifluoromethyl)phenoxy)cyclohexyl)amino)cyclobut-3-ene, 4-(((1,4-trans)-4-((2-((3-fluorophenyl)amino)-3,4-dioxocyclobut-1-en-1-yl)amino)cyclohexyl)oxy)benzonitrile, 3-((3-fluorophenyl)amino)-4-(((1,4-trans)-4-(4-nitrophenoxy)cyclohexyl)amino)cyclobut-3-ene-1,2-dione, 3-((3-fluorophenyl)amino)-4-(((1,4-trans)-4-(4-(methylsulfonyl)phenoxy)cyclohexyl)amino)cyclobut-3-ene-1,2-dione, 4-(((1,4-trans)-4-((2-((3-fluorophenyl)amino)-3,4-dioxocyclobut-1-en-1-yl)amino)cyclohexyl)oxy)benzenesulfonamide, 3-((3-chlorophenyl)amino)-4-(((1,4-trans)-4-(4-(trifluoromethyl)phenoxy)cyclohexyl)amino)cyclobut-3-ene-1,2-dione, 4-(((1,4-trans)-4-((2-((3-chlorophenyl)amino)-3,4-dioxocyclobut-1-en-1-yl)amino)cyclohexyl)oxy)benzonitrile, 3-((3-chlorophenyl)amino)-4-(((1,4-trans)-4-(4-nitrophenoxy)cyclohexyl)amino)cyclobut-3-ene-1,2-dione, 3-((3-chlorophenyl)amino)-4-(((1,4-trans)-4-(4-(methylsulfonyl)phenoxy)cyclohexyl)amino)cyclobut-3-ene-1,2-dione, 4-(((1,4-trans)-4-((2-((3-chlorophenyl)amino)-3,4-dioxocyclobut-1-en-1-yl)amino)cyclohexyl)oxy)benzenesulfonamide, 3-(((1,4-trans)-4-(4-(trifluoromethyl)phenoxy)cyclohexyl)amino)-4-((3-(trifluoromethyl)phenyl)amino)cyclobut-3-ene-1,2-dione, 4-(((1,4-trans)-4-((3,4-dioxo-2-((3-(trifluoromethyl)phenyl)amino)cyclobut-1-en-1-yl)amino)cyclohexyl)oxy)benzonitrile, 3-(((1,4-trans)-4-(4-nitrophenoxy)cyclohexyl)amino)-4-((3-(trifluoromethyl)phenyl)amino)cyclobut-3-ene-1,2-dione, 3-(((1,4-trans)-4-(4-(methylsulfonyl)phenoxy)cyclohexyl)amino)-4-((3-(trifluoromethyl)phenyl)amino)cyclobut-3-ene-1,2-dione, 4-(((1,4-trans)-4-((3,4-dioxo-2-((3-(trifluoromethyl)phenyl)amino)cyclobut-1-en-1-yl)amino)cyclohexyl)oxy)benzenesulfonamide, 3-((3-(tert-butyl)phenyl)amino)-4-(((1,4-trans)-4-(4-(trifluoromethyl)phenoxy)cyclohexyl)amino)cyclobut-3-ene-1,2-dione, 4-(((1,4-trans)-4-((2-((3-(tert-butyl)phenyl)amino)-3,4-dioxocyclobut-1-en-1-yl)amino)cyclohexyl)oxy)benzonitrile, 3-((3-(tert-butyl)phenyl)amino)-4-(((1,4-trans)-4-(4-nitrophenoxy)cyclohexyl)amino)cyclobut-3-ene-1,2-dione, 3-((3-(tert-butyl)phenyl)amino)-4-(((1,4-trans)-4-(4-(methylsulfonyl)phenoxy)cyclohexyl)amino)cyclobut-3-ene-1,2-dione, 4-(((1,4-trans)-4-((2-((3-(tert-butyl)phenyl)amino)-3,4-dioxocyclobut-1-en-1-yl)amino)cyclohexyl)oxy)benzenesulfonamide, 3-((3-methoxyphenyl)amino)-4-(((1,4-trans)-4-(4-(trifluoromethyl)phenoxy)cyclohexyl)amino)cyclobut-3-ene-1,2-dione, 4-(((1,4-trans)-4-((2-((3-methoxyphenyl)amino)-3,4-dioxocyclobut-1-en-1-yl)amino)cyclohexyl)oxy)benzonitrile, 3-((3-methoxyphenyl)amino)-4-(((1,4-trans)-4-(4-nitrophenoxy)cyclohexyl)amino)cyclobut-3-ene-1,2-dione, 3-((3-methoxyphenyl)amino)-4-(((1,4-trans)-4-(4-(methylsulfonyl)phenoxy)cyclohexyl)amino)cyclobut-3-ene-1,2-dione, 4-(((1,4-trans)-4-((2-((3-methoxyphenyl)amino)-3,4-dioxocyclobut-1-en-1-yl)amino)cyclohexyl)oxy)benzenesulfonamide, 3-((4-fluorophenyl)amino)-4-(((1,4-trans)-4-(4-(trifluoromethyl)phenoxy)cyclohexyl)amino)cyclobut-3-ene-1,2-dione, 4-(((1,4-trans)-4-((2-((4-fluorophenyl)amino)-3,4-dioxocyclobut-1-en-1-yl)amino)cyclohexyl)oxy)benzonitrile, 3-((4-fluorophenyl)amino)-4-(((1,4-trans)-4-(4-nitrophenoxy)cyclohexyl)amino)cyclobut-3-ene-1,2-dione, 3-((4-fluorophenyl)amino)-4-(((1,4-trans)-4-(4-(methylsulfonyl)phenoxy)cyclohexyl)amino)cyclobut-3-ene-1,2-dione, 3-((4-chlorophenyl)amino)-4-(((1,4-trans)-4-(4-(trifluoromethyl)phenoxy)cyclohexyl)amino)cyclobut-3-ene-1,2-dione, 4-(((1,4-trans)-4-((2-((4-chlorophenyl)amino)-3,4-dioxocyclobut-1-en-1-yl)amino)cyclohexyl)oxy)benzonitrile, 3-((4-chlorophenyl)amino)-4-(((1,4-trans)-4-(4-nitrophenoxy)cyclohexyl)amino)cyclobut-3-ene-1,2-dione, 3-((4-chlorophenyl)amino)-4-(((1,4-trans)-4-(4-(methylsulfonyl)phenoxy)cyclohexyl)amino)cyclobut-3-ene-1,2-dione, 3-(((1,4-trans)-4-(4-(trifluoromethyl)phenoxy)cyclohexyl)amino)-4-((4-(trifluoromethyl)phenyl)amino)cyclobut-3-ene-1,2-dione, 4-(((1,4-trans)-4-((3,4-dioxo-2-((4-(trifluoromethyl)phenyl)amino)cyclobut-1-en-1-yl)amino)cyclohexyl)oxy)benzonitrile, 3-(((1,4-trans)-4-(4-nitrophenoxy)cyclohexyl)amino)-4-((4-(trifluoromethyl)phenyl)amino)cyclobut-3-ene-1,2-dione, 3-(((1,4-trans)-4-(4-(methylsulfonyl)phenoxy)cyclohexyl)amino)-4-((4-(trifluoromethyl)phenyl)amino)cyclobut-3-ene-1,2-dione, 3-((4-(tert-butyl)phenyl)amino)-4-(((1,4-trans)-4-(4-(trifluoromethyl)phenoxy)cyclohexyl)amino)cyclobut-3-ene-1,2-dione, 4-(((1,4-trans)-4-((2-((4-(tert-butyl)phenyl)amino)-3,4-dioxocyclobut-1-en-1-yl)amino)cyclohexyl)oxy)benzonitrile, 3-((4-(tert-butyl)phenyl)amino)-4-(((1,4-trans)-4-(4-nitrophenoxy)cyclohexyl)amino)cyclobut-3-ene-1,2-dione, 3-((4-(tert-butyl)phenyl)amino)-4-(((1,4-trans)-4-(4-(methylsulfonyl)phenoxy)cyclohexyl)amino)cyclobut-3-ene-1,2-dione, 3-((4-methoxyphenyl)amino)-4-(((1,4-trans)-4-(4-(trifluoromethyl)phenoxy)cyclohexyl)amino)cyclobut-3-ene-1,2-dione, 4-(((1,4-trans)-4-((2-((4-methoxyphenyl)amino)-3,4-dioxocyclobut-1-en-1-yl)amino)cyclohexyl)oxy)benzonitrile, 3-((4-methoxyphenyl)amino)-4-(((1,4-trans)-4-(4-nitrophenoxy)cyclohexyl)amino)cyclobut-3-ene-1,2-dione, 3-((4-methoxyphenyl)amino)-4-(((1,4-trans)-4-(4-(methylsulfonyl)phenoxy)cyclohexyl)amino)cyclobut-3-ene-1,2-dione, methyl 4-((3,4-dioxo-2-(((1,4-trans)-4-(4-(trifluoromethyl)phenoxy)cyclohexyl)amino)cyclobut-1-en-1-yl)amino)benzoate, methyl 4-((2-(((1,4-trans)-4-(4-cyanophenoxy)cyclohexyl)amino)-3,4-dioxocyclobut-1-en-1-yl)amino)benzoate, methyl 4-((2-(((1,4-trans)-4-(4-nitrophenoxy)cyclohexyl)amino)-3,4-dioxocyclobut-1-en-1-yl)amino)benzoate, methyl 4-((2-(((1,4-trans)-4-(4-(methylsulfonyl)phenoxy)cyclohexyl)amino)-3,4-dioxocyclobut-1-en-1-yl)amino)benzoate, 3-((2-fluorophenyl)amino)-4-(((1,4-trans)-4-(4-(trifluoromethyl)phenoxy)cyclohexyl)amino)cyclobut-3-ene-1,2-dione, 4-(((1,4-trans)-4-((2-((2-fluorophenyl)amino)-3,4-dioxocyclobut-1-en-1-yl)amino)cyclohexyl)oxy)benzonitrile, 3-((2-fluorophenyl)amino)-4-(((1,4-trans)-4-(4-nitrophenoxy)cyclohexyl)amino)cyclobut-3-ene-1,2-dione, 3-((2-fluorophenyl)amino)-4-(((1,4-trans)-4-(4-(methylsulfonyl)phenoxy)cyclohexyl)amino)cyclobut-3-ene-1,2-dione, 3-((2-chlorophenyl)amino)-4-(((1,4-trans)-4-(4-(trifluoromethyl)phenoxy)cyclohexyl)amino)cyclobut-3-ene-1,2-dione, 4-(((1,4-trans)-4-((2-((2-chlorophenyl)amino)-3,4-dioxocyclobut-1-en-1-yl)amino)cyclohexyl)oxy)benzonitrile, 3-((2-chlorophenyl)amino)-4-(((1,4-trans)-4-(4-nitrophenoxy)cyclohexyl)amino)cyclobut-3-ene-1,2-dione, 3-((2-chlorophenyl)amino)-4-(((1,4-trans)-4-(4-(methylsulfonyl)phenoxy)cyclohexyl)amino)cyclobut-3-ene-1,2-dione, 3-(((1,4-trans)-4-(4-(trifluoromethyl)phenoxy)cyclohexyl)amino)-4-((2-(trifluoromethyl)phenyl)amino)cyclobut-3-ene-1,2-dione, 4-(((1,4-trans)-4-((3,4-dioxo-2-((2-(trifluoromethyl)phenyl)amino)cyclobut-1-en-1-yl)amino)cyclohexyl)oxy)benzonitrile, 3-(((1,4-trans)-4-(4-nitrophenoxy)cyclohexyl)amino)-4-((2-(trifluoromethyl)phenyl)amino)cyclobut-3-ene-1,2-dione, 3-(((1,4-trans)-4-(4-(methylsulfonyl)phenoxy)cyclohexyl)amino)-4-((2-(trifluoromethyl)phenyl)amino)cyclobut-3-ene-1,2-dione, 3-((2-(tert-butyl)phenyl)amino)-4-(((1,4-trans)-4-(4-(trifluoromethyl)phenoxy)cyclohexyl)amino)cyclobut-3-ene-1,2-dione, 4-(((1,4-trans)-4-((2-((2-(tert-butyl)phenyl)amino)-3,4-dioxocyclobut-1-en-1-yl)amino)cyclohexyl)oxy)benzonitrile, 3-((2-(tert-butyl)phenyl)amino)-4-(((1,4-trans)-4-(4-nitrophenoxy)cyclohexyl)amino)cyclobut-3-ene-1,2-dione, 3-((2-(tert-butyl)phenyl)amino)-4-(((1,4-trans)-4-(4-(methylsulfonyl)phenoxy)cyclohexyl)amino)cyclobut-3-ene-1,2-dione, 3-((2-methoxyphenyl)amino)-4-(((1,4-trans)-4-(4-(trifluoromethyl)phenoxy)cyclohexyl)amino)cyclobut-3-ene-1,2-dione, 4-(((1,4-trans)-4-((2-((2-methoxyphenyl)amino)-3,4-dioxocyclobut-1-en-1-yl)amino)cyclohexyl)oxy)benzonitrile, 3-((2-methoxyphenyl)amino)-4-(((1,4-trans)-4-(4-nitrophenoxy)cyclohexyl)amino)cyclobut-3-ene-1,2-dione, 3-((2-methoxyphenyl)amino)-4-(((1,4-trans)-4-(4-(methylsulfonyl)phenoxy)cyclohexyl)amino)cyclobut-3-ene-1,2-dione, 4-(((1,4-trans)-4-((2-((2-methoxyphenyl)amino)-3,4-dioxocyclobut-1-en-1-yl)amino)cyclohexyl)oxy)benzenesulfonamide, 3-((3,4-dioxo-2-(((1,4-trans)-4-(4-(trifluoromethyl)phenoxy)cyclohexyl)amino)cyclobut-1-en-1-yl)amino)-5-(trifluoromethyl)benzonitrile, 3-((2-(((1,4-trans)-4-(4-cyanophenoxy)cyclohexyl)amino)-3,4-dioxocyclobut-1-en-1-yl)amino)-5-(trifluoromethyl)benzonitrile, 3-((2-(((1,4-trans)-4-(4-nitrophenoxy)cyclohexyl)amino)-3,4-dioxocyclobut-1-en-1-yl)amino)-5-(trifluoromethyl)benzonitrile, and 3-((2-(((1,4-trans)-4-(4-(methylsulfonyl)phenoxy)cyclohexyl)amino)-3,4-dioxocyclobut-1-en-1-yl)amino)-5-(trifluoromethyl)benzonitrile.

In addition, the present disclosure provides a pharmaceutical composition for preventing or treating cancer, including the compound as an active ingredient.

Preferably, the compound according to the present disclosure may promote phosphorylation of eukaryotic translation initiation factor 2α (eIF2α).

Preferably, the cancer may be, but is not limited to, blood cancer, breast cancer, sarcoma cancer, or brain cancer.

In addition, the present disclosure provides a pharmaceutical composition for metabolic anticancer drugs, including the compound as an active ingredient.

In addition, the present disclosure provides a method of inhibiting cancer cell metabolism, including administering the compound.

The compound may promote phosphorylation of eukaryotic translation initiation factor 2α (eIF2α) to block the metabolism of cancer cells and cause death.

The metabolic anticancer drug is called a fourth-generation anticancer drug following chemo-anticancer drugs, targeted anticancer drugs, and immuno-oncology drugs, blocks, unlike existing anticancer drugs, the cancer cell metabolic process in a way of blocking cancer cell metabolic activity that causes cell division more actively than normal cells, and selectively kills cancer cells, and in particular, it lowers cancer cell metabolism by more than 50% with little damage to normal cells in the process of inhibiting cancer cell growth.

In the present disclosure, it is noted that the pharmaceutically acceptable salts may be, but are not limited to, one or more basic salts selected from the group consisting of sodium salts, potassium salts, calcium salts, lithium salts, magnesium salts, cesium salts, aminium salts, ammonium salts, triethylamine salts, and pyridinium salts.

In addition, the pharmaceutically acceptable salts may be, but are not limited to, one or more acidic salts selected from the group consisting of hydrochloric acid, bromic acid, sulfuric acid, sulfurous acid, phosphoric acid, citric acid, acetic acid, maleic acid, fumaric acid, gluconic acid, methanesulfonic acid, benzenesulfonic acid, camphorsulfonic acid, oxalic acid, malonic acid, glutaric acid, acetic acid, glycolic acid, succinic acid, tartaric acid, 4-toluenesulfonic acid, galacturonic acid, embonic acid, glutamic acid, citric acid, and aspartic acid.

The pharmaceutical composition of the present disclosure may include pharmaceutically acceptable carriers, excipients, or diluents in addition to the above-described components for administration. The carrier, excipient, and diluent may be lactose, dextrose, sucrose, sorbitol, mannitol, xylitol, erythritol, maltitol, starch, gum acacia, alginate, gelatin, calcium phosphate, calcium silicate, cellulose, methylcellulose, microcrystalline cellulose, polyvinylpyrrolidone, water, methylhydroxybenzoate, propylhydroxybenzoate, talc, magnesium stearate, and mineral oil.

Each of the pharmaceutical compositions of the present disclosure may be used by being formulated in the form of oral formulations such as acids, granules, tablets, capsules, suspensions, emulsions, syrups, and aerosols, topical agents, suppositories, or sterile injection solutions according to the conventional method. Specifically, when formulated, it may be prepared using diluents or excipients such as fillers, weighting agents, binders, wetting agents, disintegrating agents, and surfactants that are conventionally used. Solid preparations for oral administration include tablets, pills, acids, granules, and capsules, but are not limited thereto. Such solid preparations may be prepared by mixing at least one or more excipients in addition to the above active ingredients, such as starch, calcium carbonate, sucrose, lactose, and gelatin. In addition to simple excipients, lubricants such as magnesium stearate and talc may also be used. In addition to liquid substances and liquid paraffin for oral use, various excipients, such as wetting agents, sweeteners, aromatics, and preservatives may be added for preparation. Preparations for parenteral administration include sterilized aqueous solutions, non-aqueous solvents, suspensions, emulsions, lyophilized preparations, and suppositories. As non-aqueous solvents and suspensions, propylene glycol, polyethylene glycol, vegetable oils such as olive oil, and injectable ester such as ethyl oleate may be used. As a base of suppositories, Witepsol, Macrosol, Tween 61, cacao butter, laurin fat, and glycerogelatin may be used.

The pharmaceutical composition of the present disclosure may be prepared as an oral or parenteral preparation, and may be administered via oral, intravenous, intraventricular, intradermal, intramuscular, intraperitoneal, nasal, or epidural routes, but is not limited thereto.

Suitable dosage of the pharmaceutical composition of the present disclosure may vary depending on the condition and weight of a patient, degree of disease, drug form, and time, but may be appropriately selected by those skilled in the art, wherein the daily dosage of the composition may preferably be 0.01 mg/kg to 100 mg/kg and administration may be performed in single to several divided doses a day as needed.

In addition, the present disclosure provides a health functional food composition for preventing or ameliorating cancer, including the compound as an active ingredient.

The health functional food composition may include various nutritional supplements, vitamins, minerals (electrolytes), flavoring agents such as synthetic flavors and natural flavors, colorants and thickening agents (cheese, chocolate), pectic acid and salts thereof, alginic acid and salts thereof, organic acids, protective colloidal thickeners, pH regulators, stabilizers, preservatives, glycerin, alcohol, and carbonating agents used in carbonated beverages. It may also include pulp for the manufacture of natural fruit juices, synthetic fruit juices, and vegetable beverages. These components may be used independently or in combination with. In addition, the health functional food composition may be in any one form of meat, sausage, bread, chocolate, candy, snacks, confectionery, pizza, ramen, chewing gum, ice cream, soup, beverage, tea, functional water, drink, alcohol, and vitamin complex.

In addition, the health functional food composition may further include food additives, and the suitability as the “food additive” is determined by the standards and criteria related to corresponding items according to the general rules and general test methods of Korean Food Additives Codex approved by the Ministry of Food and Drug Safety, unless otherwise stipulated.

The items listed in the “Korean Food Additives Codex” may include, for example, chemically synthesized compounds such as ketones, glycine, potassium citrate, nicotinic acid, and cinnamic acid, natural additives such as persimmon color, licorice extracts, crystallized cellulose, kaoliang color, and guar gum, and mixed preparations such as sodium L-glutamate preparations, noodle-added alkali agents, preservative agents, and tar color agents.

In this case, a content of the composition according to the present disclosure added to the food in the process of preparing the health functional food composition may be appropriately adjusted as needed.

### MODES FOR CARRYING OUT THE INVENTION

Hereinafter, the present disclosure will be described in more detail through Examples and Experimental Examples. These Examples and Experimental Examples are only for the purpose of describing the present disclosure in more detail, and it will be apparent to those skilled in the art that the scope of the present disclosure is not limited by these Examples and Experimental Examples according to the gist of the present disclosure.

### Synthesis Example

### 1. 3-(Phenylamino)-4-(((1,4-trans)-4-(4-(trifluoromethyl)phenoxy)cyclohexyl)amino)cyclobut-3-ene-1,2-dione (Compound 8)

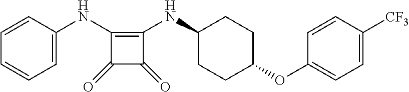 [SMILES: O=c1c(Nc2ccccc2)c(N[C@H]2CC[C@H](Oc3ccc(C(F)(F)F)cc3)CC2)c1=O]

While stirring EtOH (1 mL) solution of 3-methoxy-4-(phenylamino)cyclobut-3-ene-1,2-dione (60.0 mg, 0.30 mmol) at room temperature, (1,4-trans)-4-(4-(trifluoromethyl)phenoxy)cyclohexan-1-amine (115.0 mg, 0.44 mmol) was added under argon atmosphere, and the reaction mixture was stirred overnight. The resulting solids were filtered and washed with MeOH and EtOAc to obtain a white solid of Compound 8 (101 mg, 95%).

¹H NMR (DMSO-d₆, 500 MHz) δ 9.58 (s, 1H), 7.77 (s, 1H), 7.63 (d, 2H, J=8.3 Hz), 7.46 (d, 2H, J=7.1 Hz), 7.34 (t, 2H, J=7.5 Hz), 7.15 (d, 2H, J=8.3 Hz), 7.02 (t, 1H, J=7.2 Hz), 4.52 (m, 1H), 3.95 (m, 1H), 2.09 (m, 4H), 1.55 (m, 4H); ¹³C NMR (DMSO-d₆, 100 MHz) δ 183.6, 180.1, 168.6, 163.7, 160.3, 139.0, 129.4, 128.6, 127.0, 127.0, 126.9, 126.9, 125.9, 123.2, 122.6, 121.4, 121.1, 120.8, 120.5, 120.4, 117.9, 115.9, 74.0, 51.8, 30.8, 29.2.

### 2. 4-(((1,4-trans)-4-((3,4-Dioxo-2-(phenylamino)cyclobut-1-en-1-yl)amino)cyclohexyl)oxy)benzonitrile (Compound 9)

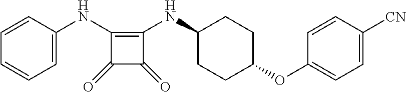 [SMILES: N#Cc1ccc(O[C@H]2CC[C@H](Nc3c(Nc4ccccc4)c(=O)c3=O)CC2)cc1]

While stirring EtOH (1 mL) solution of 3-methoxy-4-(phenylamino)cyclobut-3-ene-1,2-dione (60.0 mg, 0.30 mmol) at room temperature, 4-(((1,4-trans)-4-aminocyclohexyl)oxy)benzonitrile (95.8 mg, 0.44 mmol) was added under argon atmosphere, and the reaction mixture was stirred overnight. The resulting solids were filtered and washed with MeOH and EtOAc to obtain an ivory solid of Compound 9 (101 mg, 89%).

¹H NMR (DMSO-d₆, 500 MHz) δ 7.74 (d, 2H, J=8.5 Hz), 7.44 (d, 2H, J=7.1 Hz), 7.34 (t, 2H, J=7.5 Hz), 7.14 (d, 2H, J=8.4 Hz), 7.03 (t, 1H, J=7.2 Hz), 4.54 (m, 1H), 3.94 (m, 1H), 2.08 (m, 4H), 1.54 (m, 4H); ¹³C NMR (DMSO-d₆, 100 MHz) δ 183.7, 180.2, 168.7, 163.9, 161.1, 139.1, 134.4, 129.5, 122.9, 119.3, 118.1, 116.5, 102.7, 74.3, 51.9, 30.9, 29.3.

### 3. 3-(((1,4-trans)-4-(4-Nitrophenoxy)cyclohexyl)amino)-4-(phenylamino)cyclobut-3-ene-1,2-dione (Compound 10)

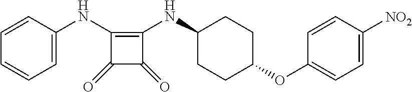 [SMILES: O=c1c(Nc2ccccc2)c(N[C@H]2CC[C@H](Oc3ccc([N+](=O)[O-])cc3)CC2)c1=O]

While stirring EtOH (1 mL) solution of 3-methoxy-4-(phenylamino)cyclobut-3-ene-1,2-dione (60.0 mg, 0.30 mmol) at room temperature, (1,4-trans)-4-(4-nitrophenoxy)cyclohexan-1-amine (105.0 mg, 0.44 mmol) was added under argon atmosphere, and the reaction mixture was stirred overnight. The resulting solids were filtered and washed with MeOH and EtOAc to obtain a light brown solid of Compound 10 (115 mg, 95%).

¹H NMR (DMSO-d₆, 500 MHz) δ 8.18 (d, 2H, J=9.3 Hz), 7.78 (s, 1H), 7.46 (d, 2H, J=7.8 Hz), 7.34 (t, 2H, J=7.9 Hz), 7.19 (d, 2H, J=9.3 Hz), 7.02 (t, 2H, J=7.4 Hz), 4.61 (m, 1H), 3.95 (m, 1H), 2.10 (m, 4H), 1.56 (m, 4H); ¹³C NMR (DMSO-d₆, 125 MHz) δ 183.6, 180.1, 168.6, 163.8, 162.9, 140.6, 139.1, 129.4, 126.0, 122.7, 118.0, 115.8, 74.8, 51.7, 30.8, 29.2.

### 4. 3-(((1,4-trans)-4-(4-(Methylsulfonyl)phenoxy)cyclohexyl)amino)-4-(phenylamino)cyclobut-3-ene-1,2-dione (Compound 11)

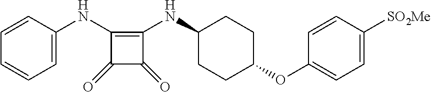 [SMILES: CS(=O)(=O)c1ccc(O[C@H]2CC[C@H](Nc3c(Nc4ccccc4)c(=O)c3=O)CC2)cc1]

While stirring EtOH (1 mL) solution of 3-methoxy-4-(phenylamino)cyclobut-3-ene-1,2-dione (50.0 mg, 0.25 mmol) at room temperature, (1,4-trans)-4-(4-(methylsulfonyl)phenoxy)cyclohexan-1-amine (99.4 mg, 0.37 mmol) was added under argon atmosphere, and the reaction mixture was stirred overnight. The resulting solids were filtered and washed with MeOH and EtOAc to obtain an ivory solid of Compound 11 (53 mg, 49%).

¹H NMR (DMSO-d₆, 500 MHz) δ 10.89 (s, 1H), 8.97 (d, 1H, J=7.6 Hz), 7.79 (d, 2H, J=8.9 Hz), 7.54 (d, 2H, J=8.0 Hz), 7.29 (t, 2H, J=7.9 Hz), 7.18 (d, 2H, J=8.9 Hz), 6.97 (t, 1H, J=7.4 Hz), 4.56 (m, 1H), 3.98 (m, 1H), 3.13 (s, 3H), 2.13 (m, 2H), 2.03 (m, 2H), 1.55 (m, 4H); ¹³C NMR (DMSO-d₆, 125 MHz) δ 183.4, 179.8, 168.8, 164.0, 161.3, 139.5, 132.3, 129.3, 129.2, 122.3, 117.8, 115.9, 74.0, 51.5, 44.0, 30.3, 28.6.

### 5. 4-(((1,4-trans)-4-((3,4-Dioxo-2-(phenylamino)cyclobut-1-en-1-yl)amino)cyclohexyl)oxy)benzenesulfonamide (Compound 12)

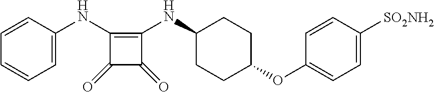 [SMILES: NS(=O)(=O)c1ccc(O[C@H]2CC[C@H](Nc3c(Nc4ccccc4)c(=O)c3=O)CC2)cc1]

While stirring EtOH (1 mL) solution of 3-methoxy-4-(phenylamino)cyclobut-3-ene-1,2-dione (40.0 mg, 0.20 mmol) at room temperature, 4-(((1,4-trans)-4-aminocyclohexyl)oxy)benzenesulfonamide (79.8 mg, 0.30 mmol) was added under argon atmosphere, and the reaction mixture was stirred overnight. The resulting solids were filtered and washed with MeOH and EtOAc to obtain an ivory solid of Compound 12 (102 mg, 99%).

¹H NMR (DMSO-d₆, 500 MHz) δ 9.57 (s, 1H), 7.74 (d, 2H, J=8.5 Hz), 7.74 (m, 1H), 7.46 (d, 2H, J=7.1 Hz), 7.34 (t, 2H, J=7.7 Hz), 7.20 (s, 2H), 7.12 (d, 2H, J=8.6 Hz), 7.03 (t, 1H, J=7.2 Hz), 4.52 (m, 1H), 3.95 (m, 1H), 2.09 (m, 4H), 1.55 (m, 4H); ¹³C NMR (DMSO-d₆, 125 MHz) δ 183.6, 180.1, 168.6, 163.7, 159.8, 139.0, 136.0, 129.4, 127.8, 122.7, 118.0, 115.4, 74.0, 51.8, 30.9, 29.2.

### 6. 3-((3-Fluorophenyl)amino)-4-(((1,4-trans)-4-(4-(trifluoromethyl)phenoxy)cyclohexyl)amino)cyclobut-3-ene (Compound 13)

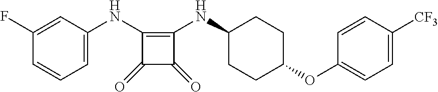 [SMILES: O=c1c(Nc2cccc(F)c2)c(N[C@H]2CC[C@H](Oc3ccc(C(F)(F)F)cc3)CC2)c1=O]

While stirring EtOH (1 mL) solution of 3-((3-fluorophenyl)amino)-4-methoxycyclobut-3-ene-1,2-dione (60.0 mg, 0.27 mmol) at room temperature, (1,4-trans)-4-(4-(trifluoromethyl)phenoxy)cyclohexan-1-amine (106.0 mg, 0.41 mmol) was added under argon atmosphere, and the reaction mixture was stirred overnight. The resulting solids were filtered and washed with MeOH and EtOAc to obtain an ivory solid of Compound 13 (90 mg, 74%).

¹H NMR (DMSO-d₆, 400 MHz) δ 9.72 (s, 1H), 7.80 (d, 1H, J=6.8 Hz), 7.62 (d, 2H, J=7.6 Hz), 7.53 (d, 1H, J=10.2 Hz), 7.36 (ddd, 1H, J=14.9, 8.2, 1.8 Hz), 7.15 (d, 2H, J=7.8 Hz), 7.12 (m, 1H), 6.82 (t, 1H, J=8.5 Hz), 4.52 (m, 1H), 3.94 (m, 1H), 2.09 (m, 4H), 1.55 (m, 4H); ¹³C NMR (DMSO-d₆, 125 MHz) δ 183.8, 180.1, 168.7, 163.8, 163.3, 161.9, 160.3, 141.0, 140.9, 131.2, 131.1, 127.9, 127.0, 127.0, 125.7, 123.6, 121.4, 121.3, 121.1, 120.8, 120.6, 115.9, 113.8, 109.0, 108.9, 105.2, 105.0, 74.0, 51.9, 30.8, 29.2.

### 7. 4-(((1,4-trans)-4-((2-((3-Fluorophenyl)amino)-3,4-dioxocyclobut-1-en-1-yl)amino)cyclohexyl)oxy)benzonitrile (Compound 14)

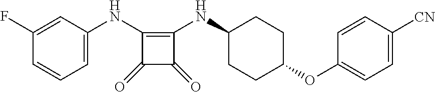 [SMILES: N#Cc1ccc(O[C@H]2CC[C@H](Nc3c(Nc4cccc(F)c4)c(=O)c3=O)CC2)cc1]

While stirring EtOH (1 mL) solution of 3-((3-fluorophenyl)amino)-4-methoxycyclobut-3-ene-1,2-dione (60.0 mg, 0.27 mmol) at room temperature, 4-(((1,4-trans)-4-aminocyclohexyl)oxy)benzonitrile (88.0 mg, 0.41 mmol) was added under argon atmosphere, and the reaction mixture was stirred overnight. The resulting solids were filtered and washed with MeOH and EtOAc to obtain an ivory solid of Compound 14 (111 mg, 99%).

¹H NMR (DMSO-d₆, 500 MHz) δ 9.63 (s, 1H), 7.76 (m, 1H), 7.75 (d, 2H, J=8.3 Hz), 7.52 (d, 1H, J=10.1 Hz), 7.36 (dd, 1H, J=14.9, 7.5 Hz), 7.14 (d, 2H, J=8.7 Hz), 7.12 (m, 1H), 6.83 (t, 1H, J=7.9 Hz), 4.55 (m, 1H), 3.94 (m, 1H), 2.08 (m, 4H), 1.54 (m, 4H); ¹³C NMR (DMSO-d₆, 125 MHz) δ 183.8, 180.1, 168.7, 163.8, 163.3, 161.9, 161.0, 141.0, 140.9, 134.3, 131.3, 131.1, 119.3, 116.4, 113.8, 109.1, 108.9, 105.2, 105.0, 102.6, 74.1, 51.9, 30.8, 29.2.

### 8. 3-((3-Fluorophenyl)amino)-4-(((1,4-trans)-4-(4-nitrophenoxy)cyclohexyl)amino)cyclobut-3-ene-1,2-dione (Compound 15)

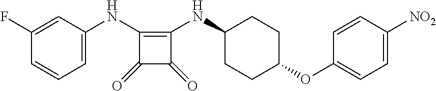 [SMILES: O=c1c(Nc2cccc(F)c2)c(N[C@H]2CC[C@H](Oc3ccc([N+](=O)[O-])cc3)CC2)c1=O]

While stirring EtOH (1 mL) solution of 3-((3-fluorophenyl)amino)-4-methoxycyclobut-3-ene-1,2-dione (60.0 mg, 0.27 mmol) at room temperature, (1,4-trans)-4-(4-nitrophenoxy)cyclohexan-1-amine (96.1 mg, 0.41 mmol) was added under argon atmosphere, and the reaction mixture was stirred overnight. The resulting solids were filtered and washed with MeOH and EtOAc to obtain a light brown solid of Compound 15 (88 mg, 76%).

¹H NMR (DMSO-d₆, 500 MHz) δ 8.19 (d, 2H, J=9.3 Hz), 7.83 (s, 1H), 7.53 (d, 1H, J=11.1 Hz), 7.36 (dd, 1H, J=15.0, 8.1 Hz), 7.19 (d, 2H, J=9.3 Hz), 7.13 (dd, 1H, J=8.1, 1.6 Hz), 6.83 (td, 1H, J=8.5, 2.2 Hz), 4.62 (m, 1H), 3.95 (m, 1H), 2.13 (m, 2H), 2.07 (m, 2H), 1.57 (m, 4H); ¹³C NMR (DMSO-d₆, 125 MHz) δ 183.8, 180.1, 168.7, 163.8, 163.3, 162.9, 161.9, 141.0, 141.0, 140.6, 131.2, 131.1, 126.0, 115.8, 113.8, 109.1, 108.9, 105.2, 105.0, 74.7, 51.8, 30.8, 29.2.

### 9. 3-((3-Fluorophenyl)amino)-4-(((1,4-trans)-4-(4-(methylsulfonyl)phenoxy)cyclohexyl)amino)cyclobut-3-ene-1,2-dione (Compound 16)

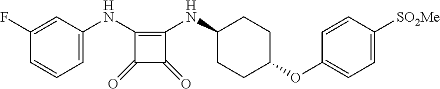 [SMILES: CS(=O)(=O)c1ccc(O[C@H]2CC[C@H](Nc3c(Nc4cccc(F)c4)c(=O)c3=O)CC2)cc1]

While stirring EtOH (1 mL) solution of 3-((3-fluorophenyl)amino)-4-methoxycyclobut-3-ene-1,2-dione (40.0 mg, 0.18 mmol) at room temperature, (1,4-trans)-4-(4-(methylsulfonyl)phenoxy)cyclohexan-1-amine (73.1 mg, 0.27 mmol) was added under argon atmosphere, and the reaction mixture was stirred overnight. The resulting solids were filtered and washed with MeOH and EtOAc to obtain an ivory solid of Compound 16 (83 mg, 99%).

¹H NMR (DMSO-d₆, 500 MHz) δ 7.82 (d, 2H, J=8.7 Hz), 7.52 (d, 1H, J=10.5), 7.36 (dd, 1H, J=15.1, 7.9 Hz), 7.19 (d, 2H, J=8.8 Hz), 7.14 (d, 1H, J=7.8 Hz), 6.83 (t, 1H, J=7.6 Hz), 4.56 (m, 1H), 3.96 (m, 1H), 3.15 (s, 3H), 2.12 (m, 2H), 2.07 (m, 2H), 1.56 (m, 4H); ¹³C NMR (DMSO-d₆, 125 MHz) δ 183.8, 180.1, 186.8, 163.8, 163.3, 161.9, 161.3, 141.1, 141.0, 132.4, 131.2, 131.1, 129.3, 115.8, 113.9, 109.1, 108.9, 105.2, 105.0, 74.2, 51.9, 44.0, 30.8, 29.2.

### 10. 4-(((1,4-trans)-4-((2-((3-Fluorophenyl)amino)-3,4-dioxocyclobut-1-en-1-yl)amino)cyclohexyl)oxy)benzenesulfonamide (Compound 17)

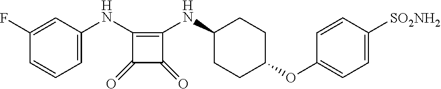 [SMILES: NS(=O)(=O)c1ccc(O[C@H]2CC[C@H](Nc3c(Nc4cccc(F)c4)c(=O)c3=O)CC2)cc1]

While stirring EtOH (1 mL) solution of 3-((3-fluorophenyl)amino)-4-methoxycyclobut-3-ene-1,2-dione (40.0 mg, 0.18 mmol) at room temperature, 4-(((1,4-trans)-4-aminocyclohexyl)oxy)benzenesulfonamide (73.3 mg, 0.27 mmol) was added under argon atmosphere, and the reaction mixture was stirred overnight. The resulting solids were filtered and washed with MeOH and EtOAc to obtain an ivory solid of Compound 17 (73 mg, 88%).

¹H NMR (DMSO-d₆, 500 MHz) δ 7.79 (s, 1H), 7.74 (d, 2H, J=8.5 Hz), 7.52 (d, 1H, J=10.0), 7.36 (dd, 1H, J=14.9, 7.6 Hz), 7.20 (s, 2H), 7.12 (d, 2H, J=8.2 Hz), 6.83 (t, 1H, J=7.7 Hz), 4.52 (m, 1H), 3.94 (m, 1H), 2.09 (m, 4H), 1.54 (m, 4H); ¹³C NMR (DMSO-d₆, 125 MHz) δ 183.8, 180.1, 168.8, 163.8, 163.3, 161.9, 159.8, 141.0, 141.0, 136.0, 131.2, 131.1, 127.8, 115.4, 113.8, 109.1, 108.9, 105.2, 105.0, 74.0, 51.9, 30.9, 29.2.

### 11. 3-((3-Chlorophenyl)amino)-4-(((1,4-trans)-4-(4-(trifluoromethyl)phenoxy)cyclohexyl)amino)cyclobut-3-ene-1,2-dione (Compound 18)

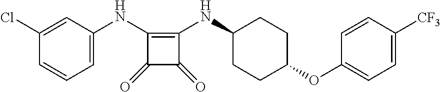 [SMILES: O=c1c(Nc2cccc(Cl)c2)c(N[C@H]2CC[C@H](Oc3ccc(C(F)(F)F)cc3)CC2)c1=O]

While stirring EtOH (1 mL) solution of 3-((3-chlorophenyl)amino)-4-methoxycyclobut-3-ene-1,2-dione (60.0 mg, 0.25 mmol) at room temperature, (1,4-trans)-4-(4-(trifluoromethyl)phenoxy)cyclohexan-1-amine (98.3 mg, 0.38 mmol) was added under argon atmosphere, and the reaction mixture was stirred overnight. The resulting solids were filtered and washed with MeOH and EtOAc to obtain a white solid of Compound 18 (109 mg, 93%).

¹H NMR (DMSO-d₆, 400 MHz) δ 9.71 (s, 1H), 7.79 (m, 1H), 7.70 (s, 1H), 7.63 (d, 2H, J=7.6 Hz), 7.34 (td, 1H, J=7.9, 1.7 Hz), 7.27 (d, 1H, 8.2 Hz), 7.15 (d, 2H, J=7.6 Hz), 7.05 (d, 1H, J=7.7 Hz), 4.52 (m, 1H), 3.94 (m, 1H), 2.09 (m, 4H), 1.54 (m, 4H); ¹³C NMR (DMSO-d₆, 100 MHz) δ 183.8, 180.1, 168.8, 163.2, 160.2, 140.6, 133.8, 130.9, 128.6, 126.9, 126.9, 125.9, 123.2, 122.1, 121.4, 121.1, 120.8, 120.5, 120.4, 117.6, 116.4, 115.8, 74.0, 51.9, 30.8, 29.2.

### 12. 4-(((1,4-trans)-4-((2-((3-Chlorophenyl)amino)-3,4-dioxocyclobut-1-en-1-yl)amino)cyclohexyl)oxy)benzonitrile (Compound 19)

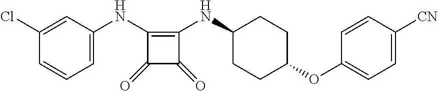 [SMILES: N#Cc1ccc(O[C@H]2CC[C@H](Nc3c(Nc4cccc(Cl)c4)c(=O)c3=O)CC2)cc1]

While stirring EtOH (1 mL) solution of 3-((3-chlorophenyl)amino)-4-methoxycyclobut-3-ene-1,2-dione (60.0 mg, 0.25 mmol) at room temperature, 4-(((1,4-trans)-4-aminocyclohexyl)oxy)benzonitrile (81.9 mg, 0.40 mmol) was added under argon atmosphere, and the reaction mixture was stirred overnight. The resulting solids were filtered and washed with MeOH and EtOAc to obtain an ivory solid of Compound 19 (99 mg, 86%).

¹H NMR (DMSO-d₆, 400 MHz) δ 7.75 (dd, 2H, J=8.6, 1.4 Hz), 7.69 (s, 1H), 7.35 (td, 1H, J=8.0, 1.2 Hz), 7.27 (d, 1H, J=7.8 Hz), 7.14 (dd, 2H, J=8.5, 1.3 Hz), 7.05 (d, 1H, J=8.0 Hz), 4.54 (m, 1H), 3.94 (m, 1H), 2.08 (m, 4H), 1.51 (m, 4H); ¹³C NMR (DMSO-d₆, 100 MHz) δ 183.8, 180.1, 168.8, 163.2, 160.9, 140.6, 134.2, 133.8, 131.0, 122.1, 119.2, 117.7, 116.4, 116.4, 102.6, 74.1, 51.8, 30.8, 29.2.

### 13. 3-((3-Chlorophenyl)amino)-4-(((1,4-trans)-4-(4-nitrophenoxy)cyclohexyl)amino)cyclobut-3-ene-1,2-dione (Compound 20)

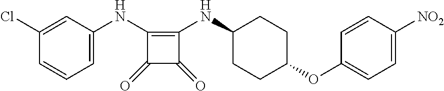 [SMILES: O=c1c(Nc2cccc(Cl)c2)c(N[C@H]2CC[C@H](Oc3ccc([N+](=O)[O-])cc3)CC2)c1=O]

While stirring EtOH (1 mL) solution of 3-((3-chlorophenyl)amino)-4-methoxycyclobut-3-ene-1,2-dione (60.9 mg, 0.26 mmol) at room temperature, (1,4-trans)-4-(4-nitrophenoxy)cyclohexan-1-amine (90.8 mg, 0.38 mmol) was added under argon atmosphere, and the reaction mixture was stirred overnight. The resulting solids were filtered and washed with MeOH and EtOAc to obtain a light brown solid of Compound 20 (78 mg, 69%).

¹H NMR (DMSO-d₆, 400 MHz) δ 9.57 (s, 1H), 8.18 (dd, 2H, J=9.1, 1.8 Hz), 7.80 (s, 1H), 7.69 (s, 1H), 7.34 (td, 1H, J=8.0, 1.4 Hz), 7.26 (d, 1H, J=8.0 Hz), 7.18 (dd, 2H, J=9.1, 1.8 Hz), 7.04 (d, 1H, J=7.8 Hz), 4.61 (m, 1H), 3.94 (m, 1H), 2.09 (m, 4H), 1.56 (m, 4H); ¹³C NMR (DMSO-d₆, 100 MHz) δ 183.8, 180.1, 168.7, 163.2, 162.8, 140.6, 133.8, 131.0, 125.9, 122.1, 117.6, 116.4, 115.7, 115.4, 74.7, 51.8, 30.7, 29.1.

### 14. 3-((3-Chlorophenyl)amino)-4-(((1,4-trans)-4-(4-(methylsulfonyl)phenoxy)cyclohexyl)amino)cyclobut-3-ene-1,2-dione (Compound 21)

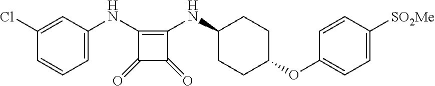 [SMILES: CS(=O)(=O)c1ccc(O[C@H]2CC[C@H](Nc3c(Nc4cccc(Cl)c4)c(=O)c3=O)CC2)cc1]

While stirring EtOH (1 mL) solution of 3-((3-chlorophenyl)amino)-4-methoxycyclobut-3-ene-1,2-dione (40.0 mg, 0.17 mmol) at room temperature, (1,4-trans)-4-(4-(methylsulfonyl)phenoxy)cyclohexan-1-amine (68.0 mg, 0.25 mmol) was added under argon atmosphere, and the reaction mixture was stirred overnight. The resulting solids were filtered and washed with MeOH and EtOAc to obtain an ivory solid of Compound 21 (71 mg, 88%).

¹H NMR (DMSO-d₆, 400 MHz) δ 7.83 (d, 2H, J=8.0 Hz), 7.70 (s, 1H), 7.34 (t, 1H, J=7.9 Hz), 7.27 (d, 1H, J=7.8 Hz), 7.19 (d, 2H, J=8.2 Hz), 7.05 (d, 1H, J=7.5 Hz), 4.56 (m, 1H), 3.95 (m, 1H), 3.16 (s, 3H), 2.09 (m, 4H), 1.55 (m, 4H); ¹³C NMR (DMSO-d₆, 100 MHz) δ 183.8, 180.1, 168.8, 163.2, 161.2, 140.6, 133.8, 132.4, 130.9, 129.2, 122.1, 117.6, 116.4, 115.8, 74.2, 51.8, 44.0, 30.7, 29.1.

### 15. 4-(((1,4-trans)-4-((2-((3-Chlorophenyl)amino)-3,4-dioxocyclobut-1-en-1-yl)amino)cyclohexyl)oxy)benzenesulfonamide (Compound 22)

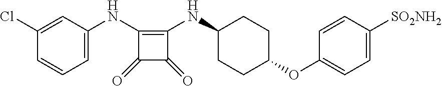 [SMILES: NS(=O)(=O)c1ccc(O[C@H]2CC[C@H](Nc3c(Nc4cccc(Cl)c4)c(=O)c3=O)CC2)cc1]

While stirring EtOH (1 mL) solution of 3-((3-chlorophenyl)amino)-4-methoxycyclobut-3-ene-1,2-dione (40.0 mg, 0.17 mmol) at room temperature, 4-(((1,4-trans)-4-aminocyclohexyl)oxy)benzenesulfonamide (68.0 mg, 0.25 mmol) was added under argon atmosphere, and the reaction mixture was stirred overnight. The resulting solids were filtered and washed with MeOH and EtOAc to obtain an ivory solid of Compound 22 (90 mg, 99%).

¹H NMR (DMSO-d₆, 500 MHz) δ 9.71 (s, 1H), 7.79 (d, 1H, J=7.2 Hz), 7.73 (d, 2H, J=8.7 Hz), 7.70 (s, 1H), 7.35 (t, 1H, J=8.2 Hz), 7.27 (d, 1H, J=8.3 Hz), 7.22 (s, 2H), 7.12 (d, 2H, J=8.7 Hz), 7.06 (d, 1H, J=7.9 Hz), 4.52 (m, 1H), 3.95 (m, 1H), 2.09 (m, 4H), 1.54 (m, 4H); ¹³C NMR (DMSO-d₆, 100 MHz) δ 183.9, 180.1, 168.8, 163.2, 159.8, 140.5, 136.0, 133.8, 131.0, 127.7, 122.1, 117.7, 116.4, 115.4, 74.0, 51.9, 30.8, 29.2.

### 16. 3-(((1,4-trans)-4-(4-(Trifluoromethyl)phenoxy)cyclohexyl)amino)-4-((3-(trifluoromethyl)phenyl)amino)cyclobut-3-ene-1,2-dione (Compound 23)

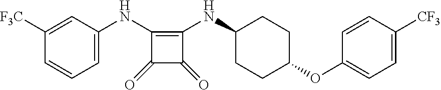 [SMILES: O=c1c(Nc2cccc(C(F)(F)F)c2)c(N[C@H]2CC[C@H](Oc3ccc(C(F)(F)F)cc3)CC2)c1=O]

While stirring EtOH (1 mL) solution of 3-methoxy-4-((3-(trifluoromethyl)phenyl)amino)cyclobut-3-ene-1,2-dione (60.0 mg, 0.22 mmol) at room temperature, (1,4-trans)-4-(4-(trifluoromethyl)phenoxy)cyclohexan-1-amine (86.0 mg, 0.33 mmol) was added under argon atmosphere, and the reaction mixture was stirred overnight. The resulting solids were filtered and washed with MeOH and EtOAc to obtain a white solid of Compound 23 (67 mg, 61%).

¹H NMR (DMSO-d₆, 500 MHz) δ 7.93 (s, 1H), 7.79 (s, 1H), 7.63 (d, 2H, J=8.6 Hz), 7.60 (m, 1H), 7.56 (t, 1H, J=7.7 Hz), 7.34 (d, 1H, J=7.2 Hz), 7.15 (d, 2H, J=8.3 Hz), 4.53 (m, 1H), 3.96 (m, 1H), 2.09 (m, 4H), 1.55 (m, 4H); ¹³C NMR (DMSO-d₆, 125 MHz) δ 184.0, 180.2, 168.9, 163.2, 160.3, 139.9, 130.6, 130.4, 130.1, 129.9, 127.9, 127.3, 127.1, 127.0, 127.0, 127.0, 125.7, 125.2, 123.6, 123.0, 121.6, 121.4, 121.3, 121.1, 120.8, 120.6, 118.7, 118.7, 115.9, 114.3, 74.0, 52.0, 30.8, 29.2.

### 17. 4-(((1,4-trans)-4-((3,4-Dioxo-2-((3-(trifluoromethyl)phenyl)amino)cyclobut-1-en-1-yl)amino)cyclohexyl)oxy)benzonitrile (Compound 24)

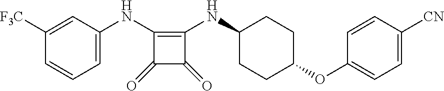 [SMILES: N#Cc1ccc(O[C@H]2CC[C@H](Nc3c(Nc4cccc(C(F)(F)F)c4)c(=O)c3=O)CC2)cc1]

While stirring EtOH (1 mL) solution of 3-methoxy-4-((3-(trifluoromethyl)phenyl)amino)cyclobut-3-ene-1,2-dione (60.0 mg, 0.22 mmol) at room temperature, 4-(((1,4-trans)-4-aminocyclohexyl)oxy)benzonitrile (71.8 mg, 0.33 mmol) was added under argon atmosphere, and the reaction mixture was stirred overnight. The resulting solids were filtered and washed with MeOH and EtOAc to obtain an ivory solid of Compound 24 (63 mg, 63%).

¹H NMR (DMSO-d₆, 500 MHz) δ 9.83 (s, 1H), 7.92 (s, 1H), 7.79 (s, 1H), 7.75 (d, 2H, J=8.6 Hz), 7.60 (d, 1H, J=8.1 Hz), 7.56 (t, 1H, J=7.8 Hz), 7.35 (d, 1H, J=7.3 Hz), 7.15 (d, 2H, J=8.7 Hz), 4.55 (m, 1H), 3.95 (m, 1H), 2.09 (m, 4H), 1.55 (m, 4H); ¹³C NMR (DMSO-d₆, 125 MHz) δ 184.0, 180.2, 168.8, 163.2, 161.0, 139.9, 134.3, 130.6, 130.4, 130.1, 129.9, 127.3, 125.1, 123.0, 121.6, 120.8, 119.2, 118.8, 118.7, 118.7, 118.7, 116.4, 114.3, 102.6, 74.1, 51.9, 30.8, 29.2.

### 18. 3-(((1,4-trans)-4-(4-Nitrophenoxy)cyclohexyl)amino)-4-((3-(trifluoromethyl)phenyl)amino)cyclobut-3-ene-1,2-dione (Compound 25)

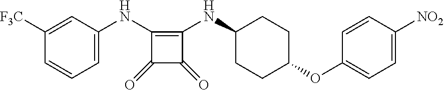 [SMILES: O=c1c(Nc2cccc(C(F)(F)F)c2)c(N[C@H]2CC[C@H](Oc3ccc([N+](=O)[O-])cc3)CC2)c1=O]

While stirring EtOH (1 mL) solution of 3-methoxy-4-((3-(trifluoromethyl)phenyl)amino)cyclobut-3-ene-1,2-dione (60.0 mg, 0.22 mmol) at room temperature, (1,4-trans)-4-(4-nitrophenoxy)cyclohexan-1-amine (78.4 mg, 0.33 mmol) was added under argon atmosphere, and the reaction mixture was stirred overnight. The resulting solids were filtered and washed with MeOH and EtOAc to obtain a reddish brown solid of Compound 25 (85 mg, 81%).

¹H NMR (DMSO-d₆, 500 MHz) δ 8.19 (d, 2H, J=9.1 Hz), 7.92 (s, 1H), 7.61 (d, 1H, J=8.0 Hz), 7.56 (t, 1H, J=7.8 Hz), 7.35 (d, 1H, J=7.4 Hz), 7.19 (d, 2H, J=9.1 Hz), 4.62 (m, 1H), 3.97 (m, 1H), 2.14 (m, 2H), 2.08 (m, 2H), 1.58 (m, 4H); ¹³C NMR (DMSO-d₆, 125 MHz) δ 184.0, 180.3, 168.9, 163.3, 162.9, 140.6, 140.0, 130.6, 130.4, 130.1, 129.9, 127.3, 126.0, 125.2, 123.0, 121.6, 120.8, 118.7, 115.8, 114.4, 74.7, 51.9, 30.8, 29.2.

### 19. 3-(((1,4-trans)-4-(4-(Methylsulfonyl)phenoxy)cyclohexyl)amino)-4-((3-(trifluoromethyl)phenyl)amino)cyclobut-3-ene-1,2-dione (Compound 26)

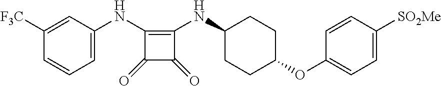 [SMILES: CS(=O)(=O)c1ccc(O[C@H]2CC[C@H](Nc3c(Nc4cccc(C(F)(F)F)c4)c(=O)c3=O)CC2)cc1]

While stirring EtOH (1 mL) solution of 3-methoxy-4-((3-(trifluoromethyl)phenyl)amino)cyclobut-3-ene-1,2-dione (40.0 mg, 0.15 mmol) at room temperature, (1,4-trans)-4-(4-(methylsulfonyl)phenoxy)cyclohexan-1-amine (59.6 mg, 0.22 mmol) was added under argon atmosphere, and the reaction mixture was stirred overnight. The resulting solids were filtered and washed with MeOH and EtOAc to obtain a reddish brown solid of Compound 26 (36 mg, 48%).

¹H NMR (DMSO-d₆, 500 MHz) δ 7.93 (s, 1H) 7.83 (d, 2H, J=8.5 Hz), 7.81 (m, 1H), 7.60 (d, 1H, J=7.8 Hz), 7.56 (t, 1H, J=7.7 Hz), 7.34 (d, 1H, J=7.2 Hz), 7.20 (d, 2H, J=8.6 Hz), 4.57 (m, 1H), 3.96 (m, 1H), 3.16 (s, 3H), 2.12 (m, 2H), 2.07 (m, 2H), 1.56 (m, 4H); ¹³C NMR (DMSO-d₆, 125 MHz) δ 184.0, 180.2, 168.9, 163.2, 161.3, 140.0, 132.4, 130.6, 130.4, 130.1, 129.9, 129.3, 127.3, 125.2, 123.0, 121.6, 120.8, 118.7, 118.7, 115.8, 114.4, 74.2, 51.9, 44.0, 30.8, 29.2.

### 20. 4-(((1,4-trans)-4-((3,4-Dioxo-2-((3-(trifluoromethyl)phenyl)amino)cyclobut-1-en-1-yl)amino)cyclohexyl)oxy)benzenesulfonamide (Compound 27)

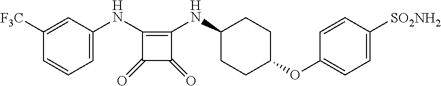 [SMILES: NS(=O)(=O)c1ccc(O[C@H]2CC[C@H](Nc3c(Nc4cccc(C(F)(F)F)c4)c(=O)c3=O)CC2)cc1]

While stirring EtOH (1 mL) solution of 3-methoxy-4-((3-(trifluoromethyl)phenyl)amino)cyclobut-3-ene-1,2-dione (40.0 mg, 0.15 mmol) at room temperature, 4-(((1,4-trans)-4-aminocyclohexyl)oxy)benzenesulfonamide (60.8 mg, 0.23 mmol) was added under argon atmosphere, and the reaction mixture was stirred overnight. The resulting solids were filtered and washed with MeOH and EtOAc to obtain an ivory solid of Compound 27 (18 mg, 23%).

¹H NMR (DMSO-d₆, 500 MHz) δ 9.84 (s, 1H), 7.93 (s, 1H) 7.73 (d, 2H, J=8.8 Hz), 7.60 (d, 1H, J=8.2 Hz), 7.56 (t, 1H, J=7.8 Hz), 7.35 (d, 1H, J=7.4 Hz), 7.12 (d, 2H, J=8.9 Hz), 4.52 (m, 1H), 3.96 (m, 1H), 2.09 (m, 4H), 1.55 (m, 4H); ¹³C NMR (DMSO-d₆, 125 MHz) δ 184.0, 180.2, 168.9, 163.2, 159.8, 139.9, 136.0, 130.6, 130.4, 130.1, 129.9, 127.8, 127.3, 125.1, 123.0, 121.5, 120.8, 118.7, 118.7, 115.4, 114.3, 74.0, 51.9, 30.8, 29.2.

### 21. 3-((3-(tert-Butyl)phenyl)amino)-4-(((1,4-trans)-4-(4-(trifluoromethyl)phenoxy)cyclohexyl)amino)cyclobut-3-ene-1,2-dione (Compound 28)

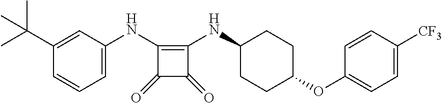 [SMILES: CC(C)(C)c1cccc(Nc2c(N[C@H]3CC[C@H](Oc4ccc(C(F)(F)F)cc4)CC3)c(=O)c2=O)c1]

While stirring EtOH (1 mL) solution of 3-((3-(tert-butyl)phenyl)amino)-4-methoxycyclobut-3-ene-1,2-dione (40.0 mg, 0.15 mmol) at room temperature, (1,4-trans)-4-(4-(trifluoromethyl)phenoxy)cyclohexan-1-amine (59.9 mg, 0.23 mmol) was added under argon atmosphere, and the reaction mixture was stirred overnight. The resulting solids were filtered and washed with MeOH and EtOAc to obtain a white solid of Compound 28 (50 mg, 67%).

¹H NMR (DMSO-d₆, 500 MHz) δ 9.54 (s, 1H), 7.69 (d, 1H, J=3.1 Hz), 7.63 (d, 2H, J=8.6 Hz), 7.53 (s, 1H), 7.26 (t, 1H, J=7.7 Hz), 7.22 (d, 1H, J=7.3 Hz), 7.16 (d, 2H, 8.5 Hz), 7.05 (d, 1H, J=7.3 Hz), 4.53 (m, 1H), 3.95 (m, 1H), 2.09 (m, 4H), 1.55 (m, 4H), 1.28 (s, 9H); ¹³C NMR (DMSO-d₆, 125 MHz) δ 183.5, 180.2, 168.5, 163.9, 160.3, 152.2, 138.7, 129.1, 127.9, 127.1, 127.0, 127.0, 127.0, 125.7, 123.6, 121.4, 121.3, 121.1, 120.8, 120.5, 119.7, 115.9, 115.3, 115.2, 74.0, 51.8, 34.6, 31.1, 30.9, 29.2.

### 22. 4-(((1,4-trans)-4-((2-((3-(tert-Butyl)phenyl)amino)-3,4-dioxocyclobut-1-en-1-yl)amino)cyclohexyl)oxy)benzonitrile (Compound 29)

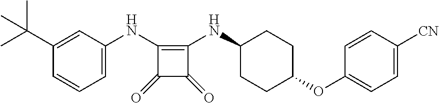 [SMILES: CC(C)(C)c1cccc(Nc2c(N[C@H]3CC[C@H](Oc4ccc(C#N)cc4)CC3)c(=O)c2=O)c1]

While stirring EtOH (1 mL) solution of 3-((3-(tert-butyl)phenyl)amino)-4-methoxycyclobut-3-ene-1,2-dione (40.0 mg, 0.15 mmol) at room temperature, 4-(((1,4-trans)-4-aminocyclohexyl)oxy)benzonitrile (50.0 mg, 0.23 mmol) was added under argon atmosphere, and the reaction mixture was stirred overnight. The resulting solids were filtered and washed with MeOH and EtOAc to obtain a white solid of Compound 29 (65 mg, 95%).

¹H NMR (DMSO-d₆, 500 MHz) δ 9.53 (s, 1H), 7.75 (d, 2H, J=8.1 Hz), 7.70 (s, 1H), 7.53 (s, 1H), 7.26 (t, 1H, J=7.7 Hz), 7.15 (d, 2H, J=8.1 Hz), 7.05 (d, 1H, J=7.3 Hz), 4.55 (m, 1H), 3.94 (m, 1H), 2.08 (m, 4H), 1.54 (m, 4H), 1.28 (s, 9H); ¹³C NMR (DMSO-d₆, 125 MHz) δ 183.5, 180.2, 168.5, 163.9, 161.0, 152.2, 138.7, 134.3, 129.1, 120.0, 119.3, 116.4, 115.2, 115.2, 102.6, 74.1, 51.7, 34.6, 31.1, 30.9, 29.2.

### 23. 3-((3-(tert-Butyl)phenyl)amino)-4-(((1,4-trans)-4-(4-nitrophenoxy)cyclohexyl)amino)cyclobut-3-ene-1,2-dione (Compound 30)

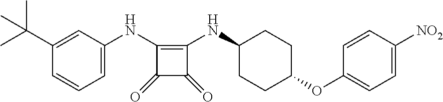 [SMILES: CC(C)(C)c1cccc(Nc2c(N[C@H]3CC[C@H](Oc4ccc([N+](=O)[O-])cc4)CC3)c(=O)c2=O)c1]

While stirring EtOH (1 mL) solution of 3-((3-(tert-butyl)phenyl)amino)-4-methoxycyclobut-3-ene-1,2-dione (50.0 mg, 0.19 mmol) at room temperature, (1,4-trans)-4-(4-nitrophenoxy)cyclohexan-1-amine (68.3 mg, 0.29 mmol) was added under argon atmosphere, and the reaction mixture was stirred overnight. The resulting solids were filtered and washed with MeOH and EtOAc to obtain a light brown solid of Compound 30 (71 mg, 78%).

¹H NMR (DMSO-d₆, 500 MHz) δ 9.55 (s, 1H), 8.19 (d, 2H, J=9.2 Hz), 7.71 (s, 1H), 7.53 (s, 1H), 7.26 (t, 1H, J=7.7 Hz), 7.19 (d, 2H, J=9.3 Hz), 7.06 (d, 1H, J=7.4 Hz), 4.62 (m, 1H), 3.96 (m, 1H), 2.13 (m, 2H), 2.08 (m, 2H), 1.57 (m, 4H), 1.28 (s, 9H); ¹³C NMR (DMSO-d₆, 125 MHz) δ 183.5, 180.2, 168.5, 163.9, 162.9, 152.2, 140.6, 138.7, 129.0, 125.9, 119.6, 115.7, 115.2, 115.1, 74.7, 51.7, 34.6, 31.0, 30.8, 29.1.

### 24. 3-((3-(tert-Butyl)phenyl)amino)-4-(((1,4-trans)-4-(4-(methylsulfonyl)phenoxy)cyclohexyl)amino)cyclobut-3-ene-1,2-dione (Compound 31)

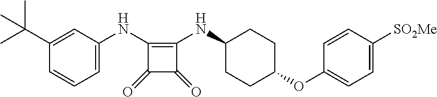 [SMILES: CC(C)(C)c1cccc(Nc2c(N[C@H]3CC[C@H](Oc4ccc(S(C)(=O)=O)cc4)CC3)c(=O)c2=O)c1]

While stirring EtOH (1 mL) solution of 3-((3-(tert-butyl)phenyl)amino)-4-methoxycyclobut-3-ene-1,2-dione (40.0 mg, 0.15 mmol) at room temperature, (1,4-trans)-4-(4-(methylsulfonyl)phenoxy)cyclohexan-1-amine (62.2 mg, 0.23 mmol) was added under argon atmosphere, and the reaction mixture was stirred overnight. The resulting solids were filtered and washed with MeOH and EtOAc to obtain a white solid of Compound 31 (63 mg, 82%).

¹H NMR (DMSO-d₆, 400 MHz) δ 7.83 (d, 2H, J=8.5 Hz), 7.74 (s, 1H), 7.53 (s, 1H), 7.26 (t, 1H, J=7.6 Hz), 7.22 (m, 1H), 7.20 (d, 2H, J=8.6 Hz), 7.06 (d, 1H, J=7.0 Hz), 4.57 (m, 1H), 3.96 (m, 1H), 3.16 (s, 3H), 2.10 (m, 4H), 1.56 (m, 4H), 1.28 (s, 9H); ¹³C NMR (DMSO-d₆, 100 MHz) δ 183.5, 180.2, 168.5, 163.9, 161.2, 152.2, 138.7, 132.4, 129.2, 129.0, 119.6, 115.8, 115.2, 115.1, 74.2, 51.7, 44.0, 34.6, 31.1, 30.8, 29.1.

### 25. 4-(((1,4-trans)-4-((2-((3-(tert-Butyl)phenyl)amino)-3,4-dioxocyclobut-1-en-1-yl)amino)cyclohexyl)oxy)benzenesulfonamide (Compound 32)

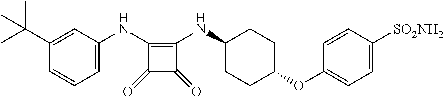 [SMILES: CC(C)(C)c1cccc(Nc2c(N[C@H]3CC[C@H](Oc4ccc(S(N)(=O)=O)cc4)CC3)c(=O)c2=O)c1]

While stirring EtOH (1 mL) solution of 3-((3-(tert-butyl)phenyl)amino)-4-methoxycyclobut-3-ene-1,2-dione (50.0 mg, 0.19 mmol) at room temperature, 4-(((1,4-trans)-4-aminocyclohexyl)oxy)benzenesulfonamide (77.8 mg, 0.29 mmol) was added under argon atmosphere, and the reaction mixture was stirred overnight. The resulting solids were filtered and washed with MeOH and EtOAc to obtain a white solid of Compound 32 (25 mg, 26%).

¹H NMR (DMSO-d₆, 500 MHz) δ 9.55 (s, 1H), 7.74 (d, 2H, J=8.8 Hz), 7.70 (s, 1H), 7.53 (s, 1H), 7.26 (t, 1H, J=7.7 Hz), 7.22 (d, 1H, J=8.4 Hz), 7.20 (s, 2H), 7.12 (d, 2H, J=8.8 Hz), 7.06 (d, 1H, J=7.4 Hz), 4.52 (m, 1H), 3.95 (m, 1H), 2.09 (m, 4H), 1.54 (m, 4H), 1.28 (s, 9H); ¹³C NMR (DMSO-d₆, 100 MHz) δ 183.5, 180.2, 168.5, 163.8, 159.7, 152.1, 138.7, 136.0, 128.9, 127.7, 119.6, 115.4, 115.2, 115.1, 74.0, 51.7, 34.5, 31.0, 30.7, 29.1.

### 26. 3-((3-Methoxyphenyl)amino)-4-(((1,4-trans)-4-(4-(trifluoromethyl)phenoxy)cyclohexyl)amino)cyclobut-3-ene-1,2-dione (Compound 33)

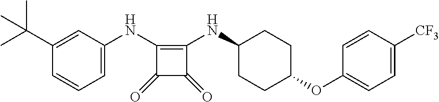 [SMILES: CC(C)(C)c1cccc(Nc2c(N[C@H]3CC[C@H](Oc4ccc(C(F)(F)F)cc4)CC3)c(=O)c2=O)c1]

While stirring EtOH (1 mL) solution of 3-methoxy-4-((3-methoxyphenyl)amino)cyclobut-3-ene-1,2-dione (60.0 mg, 0.26 mmol) at room temperature, (1,4-trans)-4-(4-(trifluoromethyl)phenoxy)cyclohexan-1-amine (100.0 mg, 0.39 mmol) was added under argon atmosphere, and the reaction mixture was stirred overnight. The resulting solids were filtered and washed with MeOH and EtOAc to obtain a white solid of Compound 33 (95 mg, 80%).

¹H NMR (DMSO-d₆, 500 MHz) δ 9.59 (s, 1H), 7.76 (s, 1H), 7.63 (d, 2H, J=8.7 Hz), 7.25 (m, 1H), 7.23 (t, 1H, J=8.0 Hz), 7.15 (d, 2H, J=8.6 Hz), 6.92 (d, 1H, J=8.4 Hz), 6.60 (dd, 1H, 8.2, 2.1 Hz), 4.52 (m, 1H), 3.95 (m, 1H), 3.75 (s, 3H), 2.09 (m, 4H), 1.55 (m, 4H); ¹³C NMR (DMSO-d₆, 100 MHz) δ 183.6, 180.1, 168.5, 163.6, 160.2, 140.2, 130.2, 128.6, 127.0, 127.0, 126.9, 126.9, 125.9, 123.2, 121.4, 121.1, 120.7, 120.5, 120.4, 115.9, 110.1, 108.4, 103.7, 74.0, 55.1, 51.8, 30.8, 29.2.

### 27. 4-(((1,4-trans)-4-((2-((3-Methoxyphenyl)amino)-3,4-dioxocyclobut-1-en-1-yl)amino)cyclohexyl)oxy)benzonitrile (Compound 34)

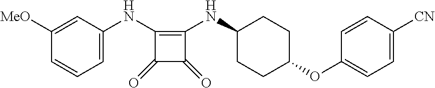 [SMILES: COc1cccc(Nc2c(N[C@H]3CC[C@H](Oc4ccc(C#N)cc4)CC3)c(=O)c2=O)c1]

While stirring EtOH (1 mL) solution of 3-methoxy-4-((3-methoxyphenyl)amino)cyclobut-3-ene-1,2-dione (60.0 mg, 0.26 mmol) at room temperature, 4-(((1,4-trans)-4-aminocyclohexyl)oxy)benzonitrile (83.5 mg, 0.36 mmol) was added under argon atmosphere, and the reaction mixture was stirred overnight. The resulting solids were filtered and washed with MeOH and EtOAc to obtain a white solid of Compound 34 (101 mg, 94%).

¹H NMR (DMSO-d₆, 500 MHz) δ 9.55 (s, 1H), 7.75 (d, 2H, J=8.5 Hz), 7.24 (m, 1H), 7.23 (t, 1H, J=7.9 Hz), 7.14 (d, 2H, J=8.6), 6.91 (d, 1H, J=7.9 Hz), 6.60 (d, 1H, J=8.2 Hz), 4.55 (m, 1H), 3.94 (m, 1H), 3.75 (s, 3H), 2.08 (m, 4H), 1.54 (m, 4H); ¹³C NMR (DMSO-d₆, 125 MHz) δ 183.6, 180.1, 168.6, 163.7, 161.0, 160.3, 140.3, 134.3, 130.3, 119.3, 116.4, 110.1, 109.4, 103.7, 102.6, 74.1, 55.1, 51.8, 30.9, 29.2.

### 28. 3-((3-Methoxyphenyl)amino)-4-(((1,4-trans)-4-(4-nitrophenoxy)cyclohexyl)amino)cyclobut-3-ene-1,2-dione (Compound 35)

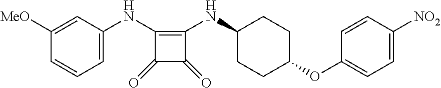 [SMILES: COc1cccc(Nc2c(N[C@H]3CC[C@H](Oc4ccc([N+](=O)[O-])cc4)CC3)c(=O)c2=O)c1]

While stirring EtOH (1 mL) solution of 3-methoxy-4-((3-methoxyphenyl)amino)cyclobut-3-ene-1,2-dione (60.0 mg, 0.26 mmol) at room temperature, (1,4-trans)-4-(4-nitrophenoxy)cyclohexan-1-amine (91.2 mg, 0.39 mmol) was added under argon atmosphere, and the reaction mixture was stirred overnight. The resulting solids were filtered and washed with MeOH and EtOAc to obtain a light brown solid of Compound 35 (99 mg, 88%).

¹H NMR (DMSO-d₆, 500 MHz) δ 8.19 (d, 2H, J=9.3 Hz), 7.79 (s, 1H), 7.24 (m, 1H), 7.23 (t, 1H, J=8.1 Hz), 7.19 (d, 2H, J=9.3 Hz), 6.92 (dd, 1H, J=7.9, 1.8 Hz), 6.60 (dd, 1H, J=8.3, 2.4 Hz), 4.62 (m, 1H), 3.96 (m, 1H), 3.76 (s, 3H), 2.13 (m, 2H), 2.08 (m, 2H), 1.57 (m, 4H); ¹³C NMR (DMSO-d₆, 100 MHz) δ 183.5, 180.1, 168.5, 163.6, 162.9, 160.2, 140.6, 140.2, 130.2, 125.9, 115.7, 110.1, 108.4, 103.7, 74.7, 55.1, 51.7, 30.8, 29.1.

### 29. 3-((3-Methoxyphenyl)amino)-4-(((1,4-trans)-4-(4-(methylsulfonyl)phenoxy)cyclohexyl)amino)cyclobut-3-ene-1,2-dione (Compound 36)

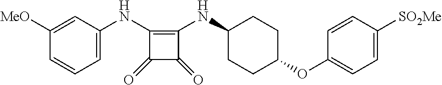 [SMILES: COc1cccc(Nc2c(N[C@H]3CC[C@H](Oc4ccc(S(C)(=O)=O)cc4)CC3)c(=O)c2=O)c1]

While stirring EtOH (1 mL) solution of 3-methoxy-4-((3-methoxyphenyl)amino)cyclobut-3-ene-1,2-dione (40.0 mg, 0.17 mmol) at room temperature, (1,4-trans)-4-(4-(methylsulfonyl)phenoxy)cyclohexan-1-amine (69.3 mg, 0.26 mmol) was added under argon atmosphere, and the reaction mixture was stirred overnight. The resulting solids were filtered and washed with MeOH and EtOAc to obtain an ivory solid of Compound 36 (74 mg, 91%).

¹H NMR (DMSO-d₆, 500 MHz) δ 7.82 (d, 2H, J=8.7 Hz), 7.75 (s, 1H), 7.25 (m, 1H), 7.23 (m, 1H), 7.19 (d, 2H, J=8.7 Hz), 6.92 (d, 1H, J=7.8 Hz), 6.60 (d, 1H, J=8.2 Hz), 4.56 (m, 1H), 3.95 (m, 1H), 3.75 (s, 3H), 3.15 (s, 3H), 2.10 (m, 4H), 1.55 (m, 4H); ¹³C NMR (DMSO-d₆, 125 MHz) δ 183.6, 180.1, 168.6, 163.7, 161.3, 160.3, 140.3, 132.4, 130.3, 129.3, 115.8, 110.1, 108.4, 103.7, 74.2, 55.1, 51.8, 44.0, 30.8, 29.2.

### 30. 4-(((1,4-trans)-4-((2-((3-Methoxyphenyl)amino)-3,4-dioxocyclobut-1-en-1-yl)amino)cyclohexyl)oxy)benzenesulfonamide (Compound 37)

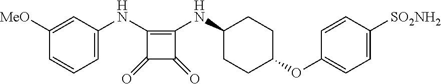 [SMILES: COc1cccc(Nc2c(N[C@H]3CC[C@H](Oc4ccc(S(N)(=O)=O)cc4)CC3)c(=O)c2=O)c1]

While stirring EtOH (1 mL) solution of 3-methoxy-4-((3-methoxyphenyl)amino)cyclobut-3-ene-1,2-dione (40.0 mg, 0.17 mmol) at room temperature, 4-(((1,4-trans)-4-aminocyclohexyl)oxy)benzenesulfonamide (69.6 mg, 0.26 mmol) was added under argon atmosphere, and the reaction mixture was stirred overnight. The resulting solids were filtered and washed with MeOH and EtOAc to obtain a white solid of Compound 37 (60 mg, 74%).

¹H NMR (DMSO-d₆, 400 MHz) δ 9.59 (s, 1H), 7.74 (d, 2H, J=8.8 Hz), 7.23 (m, 4H), 7.12 (d, 2H, J=8.8 Hz), 6.91 (dd, 1H, J=8.0, 1.6 Hz), 6.60 (dd, 1H, J=8.2, 2.2 Hz), 4.51 (m, 1H), 3.93 (m, 1H), 3.75 (s, 3H), 2.09 (m, 4H), 1.54 (m, 4H); ¹³C NMR (DMSO-d₆, 100 MHz) δ 183.6, 180.1, 168.6, 163.7, 160.3, 159.8, 140.3, 136.0, 130.3, 127.8, 115.4, 110.1, 108.4, 103.7, 74.0, 55.1, 51.8, 30.8, 29.2.

### 31. 3-((4-Fluorophenyl)amino)-4-(((1,4-trans)-4-(4-(trifluoromethyl)phenoxy)cyclohexyl)amino)cyclobut-3-ene-1,2-dione (Compound 38)

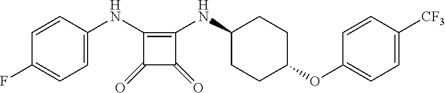 [SMILES: O=c1c(Nc2ccc(F)cc2)c(N[C@H]2CC[C@H](Oc3ccc(C(F)(F)F)cc3)CC2)c1=O]

While stirring EtOH (1 mL) solution of 3-((4-fluorophenyl)amino)-4-methoxycyclobut-3-ene-1,2-dione (40.0 mg, 0.18 mmol) at room temperature, (1,4-trans)-4-(4-(trifluoromethyl)phenoxy)cyclohexan-1-amine (70.3 mg, 0.27 mmol) was added under argon atmosphere, and the reaction mixture was stirred overnight. The resulting solids were filtered and washed with MeOH and EtOAc to obtain an ivory solid of Compound 38 (68 mg, 84%).

¹H NMR (DMSO-d₆, 400 MHz) δ 7.74 (s, 1H), 7.62 (d, 2H, J=8.6 Hz), 7.46 (dd, 2H, J=7.9, 4.1 Hz), 7.18 (m, 2H), 7.15 (d, 2H, J=9.0 Hz), 4.52 (m, 1H), 3.94 (m, 1H), 2.08 (m, 4H), 1.54 (m, 4H); ¹³C NMR (DMSO-d₆, 100 MHz) δ 183.4, 180.2, 168.5, 163.6, 160.3, 159.3, 156.9, 135.5, 135.5, 128.6, 127.0, 127.0, 126.9, 126.9, 125.9, 123.2, 121.4, 121.1, 120.8, 120.5, 120.4, 119.8, 119.7, 116.1, 115.9, 74.0, 51.8, 30.8, 29.2.

### 32. 4-(((1,4-trans)-4-((2-((4-Fluorophenyl)amino)-3,4-dioxocyclobut-1-en-1-yl)amino)cyclohexyl)oxy)benzonitrile (Compound 39)

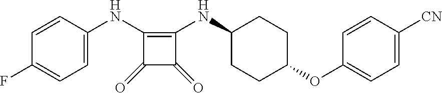 [SMILES: N#Cc1ccc(O[C@H]2CC[C@H](Nc3c(Nc4ccc(F)cc4)c(=O)c3=O)CC2)cc1]

While stirring EtOH (1 mL) solution of 3-((4-fluorophenyl)amino)-4-methoxycyclobut-3-ene-1,2-dione (40.0 mg, 0.18 mmol) at room temperature, 4-(((1,4-trans)-4-aminocyclohexyl)oxy)benzonitrile (58.7 mg, 0.27 mmol) was added under argon atmosphere, and the reaction mixture was stirred overnight. The resulting solids were filtered and washed with MeOH and EtOAc to obtain a white solid of Compound 39 (64 mg, 87%).

¹H NMR (DMSO-d₆, 500 MHz) δ 9.59 (s, 1H), 7.76 (d, 2H, J=8.8 Hz), 7.72 (s, 1H), 7.45 (m, 2H), 7.15 (d, 2H, J=8.9 Hz), 4.55 (m, 1H), 3.94 (m, 1H), 2.08 (m, 4H), 1.54 (m, 4H); ¹³C NMR (DMSO-d₆, 125 MHz) δ 183.4, 180.2, 168.5, 163.6, 161.0, 159.1, 157.2, 135.5, 135.5, 134.3, 119.8, 119.7, 119.2, 116.4, 116.1, 116.0, 102.6, 74.1, 51.8, 30.9, 29.2.

### 33. 3-((4-Fluorophenyl)amino)-4-(((1,4-trans)-4-(4-nitrophenoxy)cyclohexyl)amino)cyclobut-3-ene-1,2-dione (Compound 40)

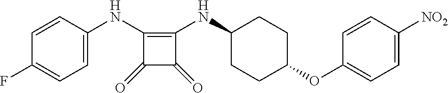 [SMILES: O=c1c(Nc2ccc(F)cc2)c(N[C@H]2CC[C@H](Oc3ccc([N+](=O)[O-])cc3)CC2)c1=O]

While stirring EtOH (1 mL) solution of 3-((4-fluorophenyl)amino)-4-methoxycyclobut-3-ene-1,2-dione (40.0 mg, 0.18 mmol) at room temperature, (1,4-trans)-4-(4-nitrophenoxy)cyclohexan-1-amine (64.0 mg, 0.27 mmol) was added under argon atmosphere, and the reaction mixture was stirred overnight. The resulting solids were filtered and washed with MeOH and EtOAc to obtain a yellow solid of Compound 40 (73 mg, 94%).

¹H NMR (DMSO-d₆, 500 MHz) δ 9.62 (s, 1H), 8.19 (d, 2H, J=9.2 Hz), 7.75 (s, 1H), 7.45 (m, 2H), 7.19 (d, 2H, J=9.1 Hz), 7.19 (m, 2H), 4.61 (m, 1H), 3.95 (m, 1H), 2.12 (m, 2H), 2.07 (m, 2H), 1.57 (m, 4H); ¹³C NMR (DMSO-d₆, 125 MHz) δ 183.4, 180.2, 168.5, 163.6, 162.9, 159.1, 157.2, 140.6, 135.5, 126.0, 119.8, 119.8, 116.1, 116.0, 115.8, 74.8, 51.7, 30.8, 29.2.

### 34. 3-((4-Fluorophenyl)amino)-4-(((1,4-trans)-4-(4-(methylsulfonyl)phenoxy)cyclohexyl)amino)cyclobut-3-ene-1,2-dione (Compound 41)

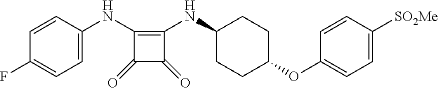 [SMILES: CS(=O)(=O)c1ccc(O[C@H]2CC[C@H](Nc3c(Nc4ccc(F)cc4)c(=O)c3=O)CC2)cc1]

While stirring EtOH (1 mL) solution of 3-((4-fluorophenyl)amino)-4-methoxycyclobut-3-ene-1,2-dione (40.0 mg, 0.18 mmol) at room temperature, (1,4-trans)-4-(4-(methylsulfonyl)phenoxy)cyclohexan-1-amine (73.1 mg, 0.27 mmol) was added under argon atmosphere, and the reaction mixture was stirred overnight. The resulting solids were filtered and washed with MeOH and EtOAc to obtain an ivory solid of Compound 41 (75 mg, 91%).

¹H NMR (DMSO-d₆, 500 MHz) δ 7.83 (d, 2H, J=8.7 Hz), 7.74 (s, 1H), 7.45 (m, 2H), 7.20 (d, 2H, J=8.7 Hz), 7.17 (m, 2H), 4.56 (m, 1H), 3.95 (m, 1H), 3.16 (s, 3H), 2.12 (m, 2H), 2.07 (m, 2H), 1.56 (m, 4H); ¹³C NMR (DMSO-d₆, 125 MHz) δ 183.4, 180.2, 168.5, 163.6, 161.3, 159.1, 157.2, 135.6, 132.4, 129.3, 119.8, 119.8, 116.2, 116.0, 115.8, 74.2, 51.8, 44.0, 30.9, 29.2.

### 35. 3-((4-Chlorophenyl)amino)-4-(((1,4-trans)-4-(4-(trifluoromethyl)phenoxy)cyclohexyl)amino)cyclobut-3-ene-1,2-dione (Compound 42)

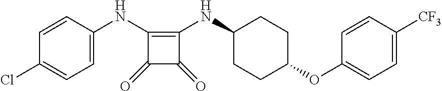 [SMILES: O=c1c(Nc2ccc(Cl)cc2)c(N[C@H]2CC[C@H](Oc3ccc(C(F)(F)F)cc3)CC2)c1=O]

While stirring EtOH (1 mL) solution of 3-((4-chlorophenyl)amino)-4-methoxycyclobut-3-ene-1,2-dione (60.0 mg, 0.25 mmol) at room temperature, (1,4-trans)-4-(4-(trifluoromethyl)phenoxy)cyclohexan-1-amine (98.2 mg, 0.38 mmol) was added under argon atmosphere, and the reaction mixture was stirred overnight. The resulting solids were filtered and washed with MeOH and EtOAc to obtain a white solid of Compound 42 (85 mg, 72%).

¹H NMR (DMSO-d₆, 500 MHz) δ 9.67 (s, 1H), 7.79 (s, 1H), 7.62 (d, 2H, J=8.3 Hz), 7.47 (d, 2H, J=7.6 Hz), 7.37 (d, 2H, J=8.5 Hz), 7.14 (d, 2H, J=8.3 Hz), 4.51 (m, 1H), 3.95 (m, 1H), 2.08 (m, 4H), 1.54 (m, 4H); ¹³C NMR (DMSO-d₆, 125 MHz) δ 183.7, 180.1, 168.7, 163.4, 160.3, 138.1, 129.3, 127.9, 127.1, 127.0, 127.0, 127.0, 126.5, 125.7, 123.6, 121.4, 121.3, 121.1, 120.8, 120.6, 119.6, 115.9, 74.0, 51.9, 30.9, 29.2.

### 36. 4-(((1,4-trans)-4-((2-((4-Chlorophenyl)amino)-3,4-dioxocyclobut-1-en-1-yl)amino)cyclohexyl)oxy)benzonitrile (Compound 43)

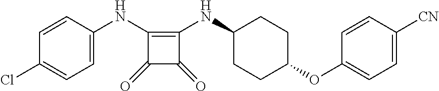 [SMILES: N#Cc1ccc(O[C@H]2CC[C@H](Nc3c(Nc4ccc(Cl)cc4)c(=O)c3=O)CC2)cc1]

While stirring EtOH (1 mL) solution of 3-((4-chlorophenyl)amino)-4-methoxycyclobut-3-ene-1,2-dione (60.0 mg, 0.25 mmol) at room temperature, 4-(((1,4-trans)-4-aminocyclohexyl)oxy)benzonitrile (82.0 mg, 0.38 mmol) was added under argon atmosphere, and the reaction mixture was stirred overnight. The resulting solids were filtered and washed with MeOH and EtOAc to obtain a light brown solid of Compound 43 (98 mg, 92%).

¹H NMR (DMSO-d₆, 500 MHz) δ 7.75 (d, 2H, J=8.0 Hz), 7.46 (d, 2H, J=6.6 Hz), 7.38 (d, 2H, J=7.2 Hz), 7.15 (d, 2H, J=8.0 Hz), 4.55 (m, 1H), 3.94 (m, 1H), 2.08 (m, 4H), 1.54 (m, 4H); ¹³C NMR (DMSO-d₆, 150 MHz) δ 183.7, 180.1, 168.6, 163.3, 160.9, 138.0, 134.2, 129.2, 126.5, 119.6, 119.2, 116.3, 102.6, 74.1, 51.8, 30.8, 29.1.

### 37. 3-((4-Chlorophenyl)amino)-4-(((1,4-trans)-4-(4-nitrophenoxy)cyclohexyl)amino)cyclobut-3-ene-1,2-dione (Compound 44)

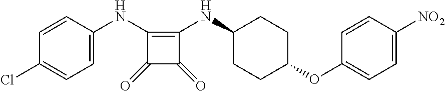 [SMILES: O=c1c(Nc2ccc(Cl)cc2)c(N[C@H]2CC[C@H](Oc3ccc([N+](=O)[O-])cc3)CC2)c1=O]

While stirring EtOH (1 mL) solution of 3-((4-chlorophenyl)amino)-4-methoxycyclobut-3-ene-1,2-dione (50.0 mg, 0.21 mmol) at room temperature, (1,4-trans)-4-(4-nitrophenoxy)cyclohexan-1-amine (74.6 mg, 0.32 mmol) was added under argon atmosphere, and the reaction mixture was stirred overnight. The resulting solids were filtered and washed with MeOH and EtOAc to obtain a brown solid of Compound 44 (69 mg, 74%).

¹H NMR (DMSO-d₆, 500 MHz) δ 10.93 (s, 1H), 8.88 (d, 1H, J=7.5 Hz), 8.18 (d, 2H, J=9.2 Hz), 7.57 (d, 2H, J=8.5 Hz), 7.36 (d, 2H, J=8.8 Hz), 7.20 (d, 2H, J=9.3 Hz), 4.63 (m, 1H), 3.99 (m, 1H), 2.10 (m, 2H), 2.05 (m, 2H), 1.58 (m, 4H); ¹³C NMR (DMSO-d₆, 125 MHz) δ 183.5, 179.8, 168.9, 163.6, 162.9, 140.5, 138.6, 129.1, 126.2, 125.9, 119.3, 115.8, 74.5, 51.5, 30.3, 28.6.

### 38. 3-((4-Chlorophenyl)amino)-4-(((1,4-trans)-4-(4-(methylsulfonyl)phenoxy)cyclohexyl)amino)cyclobut-3-ene-1,2-dione (Compound 45)

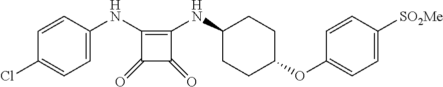 [SMILES: CS(=O)(=O)c1ccc(O[C@H]2CC[C@H](Nc3c(Nc4ccc(Cl)cc4)c(=O)c3=O)CC2)cc1]

While stirring EtOH (1 mL) solution of 3-((4-chlorophenyl)amino)-4-methoxycyclobut-3-ene-1,2-dione (40.0 mg, 0.17 mmol) at room temperature, (1,4-trans)-4-(4-(methylsulfonyl)phenoxy)cyclohexan-1-amine (68.0 mg, 0.25 mmol) was added under argon atmosphere, and the reaction mixture was stirred overnight. The resulting solids were filtered and washed with MeOH and EtOAc to obtain a light yellow solid of Compound 45 (70 mg, 88%).

¹H NMR (DMSO-d₆, 500 MHz) δ 7.82 (d, 2H, J=8.5 Hz), 7.46 (d, 2H, J=7.7 Hz), 7.38 (d, 2H, J=8.2 Hz), 7.19 (d, 2H, J=8.4 Hz), 4.56 (m, 1H), 3.95 (m, 1H), 3.15 (s, 3H), 2.12 (m, 2H), 2.07 (m, 2H), 1.55 (m, 4H); ¹³C NMR (DMSO-d₆, 125 MHz) δ 183.7, 180.1, 168.7, 163.4, 161.3, 138.1, 132.4, 129.3, 129.3, 126.5, 119.6, 115.8, 74.2, 51.8, 44.0, 30.8, 29.2.

### 39. 3-(((1,4-trans)-4-(4-(Trifluoromethyl)phenoxy)cyclohexyl)amino)-4-((4-(trifluoromethyl)phenyl)amino)cyclobut-3-ene-1,2-dione (Compound 46)

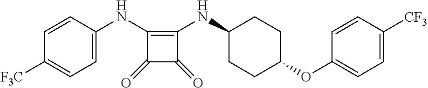 [SMILES: O=c1c(Nc2ccc(C(F)(F)F)cc2)c(N[C@H]2CC[C@H](Oc3ccc(C(F)(F)F)cc3)CC2)c1=O]

While stirring EtOH (1 mL) solution of 3-methoxy-4-((4-(trifluoromethyl)phenyl)amino)cyclobut-3-ene-1,2-dione (50.0 mg, 0.18 mmol) at room temperature, (1,4-trans)-4-(4-(trifluoromethyl)phenoxy)cyclohexan-1-amine (71.7 mg, 0.28 mmol) was added under argon atmosphere, and the reaction mixture was stirred overnight. The resulting solids were filtered and washed with MeOH and EtOAc to obtain an ivory solid of Compound 46 (63 mg, 68%).

¹H NMR (DMSO-d₆, 500 MHz) δ 9.87 (s, 1H), 7.84 (d, 2H, J=6.4 Hz), 7.68 (d, 2H, J=8.6 Hz), 7.62 (m, 4H), 7.15 (d, 2H, J=8.6 Hz), 4.52 (m, 1H), 3.96 (m, 1H), 2.09 (m, 4H), 1.55 (m, 4H); ¹³C NMR (DMSO-d₆, 125 MHz) δ 184.3, 180.0, 169.1, 163.1, 160.3, 142.6, 127.8, 127.7, 127.0, 127.0, 127.0, 126.9, 126.7, 126.7, 125.7, 125.6, 123.5, 123.4, 122.8, 122.5, 122.3, 122.0, 121.4, 121.3, 121.3, 121.1, 120.8, 120.6, 117.9, 115.9, 74.0, 51.9, 30.8, 29.2.

### 40. 4-(((1,4-trans)-4-((3,4-Dioxo-2-((4-(trifluoromethyl)phenyl)amino)cyclobut-1-en-1-yl)amino)cyclohexyl)oxy)benzonitrile (Compound 47)

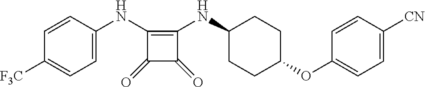 [SMILES: N#Cc1ccc(O[C@H]2CC[C@H](Nc3c(Nc4ccc(C(F)(F)F)cc4)c(=O)c3=O)CC2)cc1]

While stirring EtOH (1 mL) solution of 3-methoxy-4-((4-(trifluoromethyl)phenyl)amino)cyclobut-3-ene-1,2-dione (50.0 mg, 0.18 mmol) at room temperature, 4-(((1,4-trans)-4-aminocyclohexyl)oxy)benzonitrile (59.8 mg, 0.28 mmol) was added under argon atmosphere, and the reaction mixture was stirred overnight. The resulting solids were filtered and washed with MeOH and EtOAc to obtain an ivory solid of Compound 47 (78 mg, 93%).

¹H NMR (DMSO-d₆, 500 MHz) δ 7.83 (s, 1H), 7.75 (d, 2H, J=8.7 Hz), 7.68 (d, 2H, J=8.7 Hz), 7.62 (d, 2H, J=8.0 Hz), 7.14 (d, 2H, J=8.8 Hz), 4.55 (m, 1H), 3.95 (m, 1H), 2.09 (m, 4H), 1.55 (m, 4H); ¹³C NMR (DMSO-d₆, 125 MHz) δ 184.3, 180.0, 169.0, 163.1, 160.9, 142.6, 134.2, 127.7, 126.7, 126.7, 125.6, 123.4, 122.7, 122.5, 122.2, 122.0, 121.3, 119.2, 117.9, 116.3, 102.6, 74.1, 51.9, 30.8, 29.2.

### 41. 3-(((1,4-trans)-4-(4-Nitrophenoxy)cyclohexyl)amino)-4-((4-(trifluoromethyl)phenyl)amino)cyclobut-3-ene-1,2-dione (Compound 48)

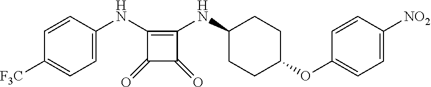 [SMILES: O=c1c(Nc2ccc(C(F)(F)F)cc2)c(N[C@H]2CC[C@H](Oc3ccc([N+](=O)[O-])cc3)CC2)c1=O]

While stirring EtOH (1 mL) solution of 3-methoxy-4-((4-(trifluoromethyl)phenyl)amino)cyclobut-3-ene-1,2-dione (50.0 mg, 0.18 mmol) at room temperature, (1,4-trans)-4-(4-nitrophenoxy)cyclohexan-1-amine (65.3 mg, 0.28 mmol) was added under argon atmosphere, and the reaction mixture was stirred overnight. The resulting solids were filtered and washed with MeOH and EtOAc to obtain a brown solid of Compound 48 (71 mg, 82%).

¹H NMR (DMSO-d₆, 500 MHz) δ 9.88 (s, 1H), 8.20 (d, 2H, J=8.8 Hz), 7.86 (s, 1H), 7.69 (d, 2H, J=8.2 Hz), 7.62 (d, 2H, J=6.8 Hz), 7.19 (d, 2H, J=8.9 Hz), 4.62 (m, 1H), 3.97 (m, 1H), 2.14 (m, 2H), 2.08 (m, 2H), 1.58 (m, 4H); ¹³C NMR (DMSO-d₆, 125 MHz) δ 184.3, 180.0, 169.1, 163.1, 162.9, 142.6, 140.6, 127.7, 126.7, 126.0, 125.6, 123.4, 122.8, 122.5, 122.2, 122.0, 121.3, 118.0, 115.8, 74.7, 51.9, 30.7, 29.1.

### 42. 3-(((1,4-trans)-4-(4-(Methylsulfonyl)phenoxy)cyclohexyl)amino)-4-((4-(trifluoromethyl)phenyl)amino)cyclobut-3-ene-1,2-dione (Compound 49)

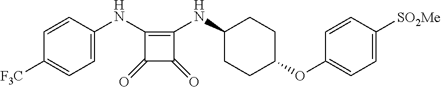 [SMILES: CS(=O)(=O)c1ccc(O[C@H]2CC[C@H](Nc3c(Nc4ccc(C(F)(F)F)cc4)c(=O)c3=O)CC2)cc1]

While stirring EtOH (1 mL) solution of 3-methoxy-4-((4-(trifluoromethyl)phenyl)amino)cyclobut-3-ene-1,2-dione (40.0 mg, 0.15 mmol) at room temperature, (1,4-trans)-4-(4-(methylsulfonyl)phenoxy)cyclohexan-1-amine (60.0 mg, 0.22 mmol) was added under argon atmosphere, and the reaction mixture was stirred overnight. The resulting solids were filtered and washed with MeOH and EtOAc to obtain a light yellow solid of Compound 49 (60 mg, 79%).

¹H NMR (DMSO-d₆, 500 MHz) δ 7.88 (s, 1H), 7.83 (d, 2H, J=8.8 Hz), 7.68 (d, 2H, J=8.7 Hz), 7.62 (d, 2H, J=7.9 Hz), 7.20 (d, 2H, J=8.8 Hz), 4.57 (m, 1H), 3.96 (m, 1H), 3.16 (s, 3H), 2.12 (m, 2H), 2.07 (m, 2H), 1.56 (m, 4H); ¹³C NMR (DMSO-d₆, 125 MHz) δ 184.3, 180.1, 169.1, 163.1, 161.3, 142.7, 132.4, 129.3, 127.8, 126.8, 126.7, 125.6, 123.5, 122.7, 122.5, 122.2, 122.0, 121.3, 118.0, 115.8, 74.2, 51.9, 44.0, 30.8, 29.2.

### 43. 3-((4-(tert-Butyl)phenyl)amino)-4-(((1,4-trans)-4-(4-(trifluoromethyl)phenoxy)cyclohexyl)amino)cyclobut-3-ene-1,2-dione (Compound 50)

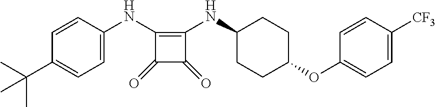 [SMILES: CC(C)(C)c1ccc(Nc2c(N[C@H]3CC[C@H](Oc4ccc(C(F)(F)F)cc4)CC3)c(=O)c2=O)cc1]

While stirring EtOH (1 mL) solution of 3-((4-(tert-butyl)phenyl)amino)-4-methoxycyclobut-3-ene-1,2-dione (40.0 mg, 0.15 mmol) at room temperature, (1,4-trans)-4-(4-(trifluoromethyl)phenoxy)cyclohexan-1-amine (59.9 mg, 0.23 mmol) was added under argon atmosphere, and the reaction mixture was stirred overnight. The resulting solids were filtered and washed with MeOH and EtOAc to obtain a white solid of Compound 50 (65 mg, 87%).

¹H NMR (DMSO-d₆, 500 MHz) δ 9.54 (s, 1H), 7.72 (s, 1H), 7.63 (d, 2H, J=8.3 Hz), 7.36 (m, 4H), 7.15 (d, 2H, J=8.2 Hz), 4.52 (m, 1H), 3.94 (m, 1H), 2.09 (m, 4H), 1.54 (m, 4H), 1.26 (s, 9H); ¹³C NMR (DMSO-d₆, 125 MHz) δ 183.4, 180.1, 168.4, 163.8, 160.3, 145.1, 136.5, 127.9, 127.1, 127.1, 127.0, 127.0, 126.1, 125.7, 123.6, 121.4, 121.3, 121.0, 120.8, 120.5, 117.8, 115.9, 74.0, 51.8, 34.1, 31.2, 30.9, 29.2.

### 44. 4-(((1,4-trans)-4-((2-((4-(tert-Butyl)phenyl)amino)-3,4-dioxocyclobut-1-en-1-yl)amino)cyclohexyl)oxy)benzonitrile (Compound 51)

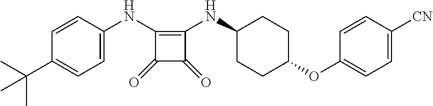 [SMILES: CC(C)(C)c1ccc(Nc2c(N[C@H]3CC[C@H](Oc4ccc(C#N)cc4)CC3)c(=O)c2=O)cc1]

While stirring EtOH (1 mL) solution of 3-((4-(tert-butyl)phenyl)amino)-4-methoxycyclobut-3-ene-1,2-dione (40.0 mg, 0.15 mmol) at room temperature, 4-(((1,4-trans)-4-aminocyclohexyl)oxy)benzonitrile (50.0 mg, 0.23 mmol) was added under argon atmosphere, and the reaction mixture was stirred overnight. The resulting solids were filtered and washed with MeOH and EtOAc to obtain a white solid of Compound 51 (60 mg, 88%).

¹H NMR (DMSO-d₆, 500 MHz) δ 9.54 (s, 1H), 7.76 (d, 2H, J=8.7 Hz), 7.72 (s, 1H), 7.36 (m, 4H), 7.15 (d, 2H, J=8.8 Hz), 4.55 (m, 1H), 3.93 (m, 1H), 2.08 (m, 4H), 1.54 (m, 4H), 1.26 (s, 9H); ¹³C NMR (DMSO-d₆, 100 MHz) δ 183.3, 180.1, 168.3, 163.8, 160.9, 145.1, 136.4, 134.2, 126.0, 119.2, 117.8, 116.3, 102.6, 74.1, 51.7, 34.0, 31.2, 30.8, 29.2.

### 45. 3-((4-(tert-Butyl)phenyl)amino)-4-(((1,4-trans)-4-(4-nitrophenoxy)cyclohexyl)amino)cyclobut-3-ene-1,2-dione (Compound 52)

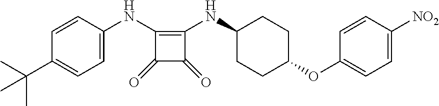 [SMILES: CC(C)(C)c1ccc(Nc2c(N[C@H]3CC[C@H](Oc4ccc([N+](=O)[O-])cc4)CC3)c(=O)c2=O)cc1]

While stirring EtOH (1 mL) solution of 3-((4-(tert-butyl)phenyl)amino)-4-methoxycyclobut-3-ene-1,2-dione (40.0 mg, 0.15 mmol) at room temperature, (1,4-trans)-4-(4-nitrophenoxy)cyclohexan-1-amine (54.7 mg, 0.23 mmol) was added under argon atmosphere, and the reaction mixture was stirred overnight. The resulting solids were filtered and washed with MeOH and EtOAc to obtain a light yellow solid of Compound 52 (62 mg, 87%).

¹H NMR (DMSO-d₆, 500 MHz) δ 9.54 (s, 1H), 8.19 (d, 2H, J=9.2 Hz), 7.73 (s, 1H), 7.36 (m, 4H), 7.19 (d, 2H, J=9.2 Hz), 4.62 (m, 1H), 3.95 (m, 1H), 2.10 (m, 4H), 1.57 (m, 4H), 1.26 (s, 9H); ¹³C NMR (DMSO-d₆, 125 MHz) δ 183.3, 180.1, 168.4, 163.8, 162.9, 145.1, 140.6, 136.5, 126.1, 126.0, 117.8, 115.8, 74.8, 51.7, 34.0, 31.2, 30.8, 29.2.

### 46. 3-((4-(tert-Butyl)phenyl)amino)-4-(((1,4-trans)-4-(4-(methylsulfonyl)phenoxy)cyclohexyl)amino)cyclobut-3-ene-1,2-dione (Compound 53)

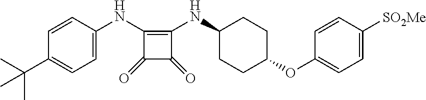 [SMILES: CC(C)(C)c1ccc(Nc2c(N[C@H]3CC[C@H](Oc4ccc(S(C)(=O)=O)cc4)CC3)c(=O)c2=O)cc1]

While stirring EtOH (1 mL) solution of 3-((4-(tert-butyl)phenyl)amino)-4-methoxycyclobut-3-ene-1,2-dione (40.0 mg, 0.15 mmol) at room temperature, (1,4-trans)-4-(4-(methylsulfonyl)phenoxy)cyclohexan-1-amine (62.3 mg, 0.23 mmol) was added under argon atmosphere, and the reaction mixture was stirred overnight. The resulting solids were filtered and washed with MeOH and EtOAc to obtain a light brown solid of Compound 53 (57 mg, 74%).

¹H NMR (DMSO-d₆, 500 MHz) δ 9.55 (s, 1H), 7.82 (d, 2H, J=8.6 Hz), 7.73 (d, 1H, J=4.0 Hz), 7.36 (m, 4H), 7.19 (d, 2H, J=8.7 Hz), 4.56 (m, 1H), 3.94 (m, 1H), 3.35 (s, 3H), 2.09 (m, 4H), 1.55 (m, 4H), 1.26 (s, 9H); ¹³C NMR (DMSO-d₆, 125 MHz) δ 183.4, 180.1, 168.4, 163.8, 161.3, 145.1, 136.5, 132.4, 129.3, 126.1, 117.8, 115.8, 74.2, 51.7, 44.0, 34.0, 31.2, 30.8, 29.2.

### 47. 3-((4-Methoxyphenyl)amino)-4-(((1,4-trans)-4-(4-(trifluoromethyl)phenoxy)cyclohexyl)amino)cyclobut-3-ene-1,2-dione (Compound 54)

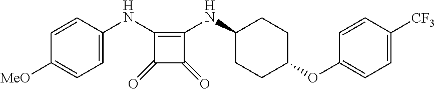 [SMILES: COc1ccc(Nc2c(N[C@H]3CC[C@H](Oc4ccc(C(F)(F)F)cc4)CC3)c(=O)c2=O)cc1]

While stirring EtOH (1 mL) solution of 3-methoxy-4-((4-methoxyphenyl)amino)cyclobut-3-ene-1,2-dione (40.0 mg, 0.17 mmol) at room temperature, (1,4-trans)-4-(4-(trifluoromethyl)phenoxy)cyclohexan-1-amine (66.6 mg, 0.26 mmol) was added under argon atmosphere, and the reaction mixture was stirred overnight. The resulting solids were filtered and washed with MeOH and EtOAc to obtain a white solid of Compound 54 (72 mg, 91%).

¹H NMR (DMSO-d₆, 500 MHz) δ 9.45 (s, 1H), 7.63 (d, 2H, J=8.7 Hz), 7.37 (d, 2H, J=8.6 Hz), 7.15 (d, 2H, J=8.7 Hz), 6.92 (d, 2H, J=9.1 Hz), 4.52 (m, 1H), 3.93 (m, 1H), 2.09 (m, 4H), 1.54 (m, 4H); ¹³C NMR (DMSO-d₆, 125 MHz) δ 183.0, 180.3, 168.2, 163.8, 160.3, 155.3, 132.3, 127.9, 127.1, 127.0, 127.0, 127.0, 125.7, 123.6, 121.4, 121.3, 121.1, 120.8, 120.5, 119.6, 115.9, 114.6, 74.0, 55.3, 51.7, 30.9, 29.2.

### 48. 4-(((1,4-trans)-4-((2-((4-Methoxyphenyl)amino)-3,4-dioxocyclobut-1-en-1-yl)amino)cyclohexyl)oxy)benzonitrile (Compound 55)

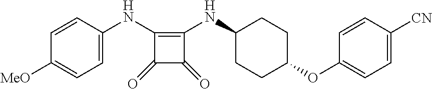 [SMILES: COc1ccc(Nc2c(N[C@H]3CC[C@H](Oc4ccc(C#N)cc4)CC3)c(=O)c2=O)cc1]

While stirring EtOH (1 mL) solution of 3-methoxy-4-((4-methoxyphenyl)amino)cyclobut-3-ene-1,2-dione (40.0 mg, 0.17 mmol) at room temperature, 4-(((1,4-trans)-4-aminocyclohexyl)oxy)benzonitrile (55.6 mg, 0.26 mmol) was added under argon atmosphere, and the reaction mixture was stirred overnight. The resulting solids were filtered and washed with MeOH and EtOAc to obtain an ivory solid of Compound 55 (62 mg, 86%).

¹H NMR (DMSO-d₆, 400 MHz) δ 9.46 (s, 1H), 7.75 (d, 2H, J=7.6 Hz), 7.66 (s, 1H), 7.36 (d, 2H, J=8.0 Hz), 7.14 (d, 2H, J=7.7 Hz), 6.92 (d, 2H, J=7.6 Hz), 4.54 (m, 1H), 3.93 (m, 1H), 3.73 (s, 3H), 2.08 (m, 4H), 1.53 (m, 4H); ¹³C NMR (DMSO-d₆, 100 MHz) δ 180.2, 168.1, 163.8, 160.9, 155.2, 134.3, 132.2, 119.6, 119.2, 116.4, 114.6, 114.3, 102.6, 74.2, 55.3, 51.7, 30.8, 29.2.

### 49. 3-((4-Methoxyphenyl)amino)-4-(((1,4-trans)-4-(4-nitrophenoxy)cyclohexyl)amino)cyclobut-3-ene-1,2-dione (Compound 56)

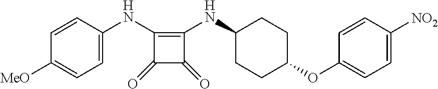 [SMILES: COc1ccc(Nc2c(N[C@H]3CC[C@H](Oc4ccc([N+](=O)[O-])cc4)CC3)c(=O)c2=O)cc1]

While stirring EtOH (1 mL) solution of 3-methoxy-4-((4-methoxyphenyl)amino)cyclobut-3-ene-1,2-dione (40.0 mg, 0.17 mmol) at room temperature, (1,4-trans)-4-(4-nitrophenoxy)cyclohexan-1-amine (60.8 mg, 0.26 mmol) was added under argon atmosphere, and the reaction mixture was stirred overnight. The resulting solids were filtered and washed with MeOH and EtOAc to obtain a light brown solid of Compound 56 (65 mg, 88%).

¹H NMR (DMSO-d₆, 400 MHz) δ 9.47 (s, 1H), 8.18 (d, 2H, J=9.2 Hz), 7.65 (s, 1H), 7.37 (d, 2H, J=8.7 Hz), 7.18 (d, 2H, J=9.2 Hz), 6.92 (d, 2H, J=8.9 Hz), 4.61 (m, 1H), 3.94 (m, 1H), 3.72 (s, 3H), 2.10 (m, 4H), 1.56 (m, 4H); ¹³C NMR (DMSO-d₆, 100 MHz) δ 183.0, 180.3, 168.1, 163.8, 162.9, 155.2, 140.6, 132.2, 125.9, 119.5, 115.7, 114.6, 74.8, 55.3, 51.6, 30.8, 29.1.

### 50. 3-((4-Methoxyphenyl)amino)-4-(((1,4-trans)-4-(4-(methylsulfonyl)phenoxy)cyclohexyl)amino)cyclobut-3-ene-1,2-dione (Compound 57)

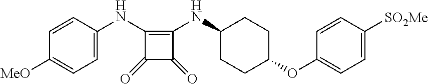 [SMILES: COc1ccc(Nc2c(N[C@H]3CC[C@H](Oc4ccc(S(C)(=O)=O)cc4)CC3)c(=O)c2=O)cc1]

While stirring EtOH (1 mL) solution of 3-methoxy-4-((4-methoxyphenyl)amino)cyclobut-3-ene-1,2-dione (40.0 mg, 0.17 mmol) at room temperature, (1,4-trans)-4-(4-(methylsulfonyl)phenoxy)cyclohexan-1-amine (69.3 mg, 0.26 mmol) was added under argon atmosphere, and the reaction mixture was stirred overnight. The resulting solids were filtered and washed with MeOH and EtOAc to obtain a white solid of Compound 57 (71 mg, 88%).

¹H NMR (DMSO-d₆, 500 MHz) δ 9.46 (s, 1H), 7.82 (d, 2H, J=8.8 Hz), 7.65 (s, 1H), 7.37 (d, 2H, J=7.9 Hz), 7.19 (d, 2H, J=8.5 Hz), 6.92 (d, 2H, J=8.8 Hz), 4.56 (m, 1H), 3.94 (m, 1H), 3.73 (s, 3H), 3.15 (s, 3H), 2.09 (m, 4H), 1.55 (m, 4H); ¹³C NMR (DMSO-d₆, 125 MHz) δ 183.0, 180.3, 168.2, 163.8, 161.3, 155.3, 132.4, 132.2, 129.3, 119.6, 115.8, 114.6, 74.2, 55.3, 51.7, 44.0, 30.9, 29.2.

### 51. Methyl 4-((3,4-dioxo-2-(((1,4-trans)-4-(4-(trifluoromethyl)phenoxy)cyclohexyl)amino)cyclobut-1-en-1-yl)amino)benzoate (Compound 58)

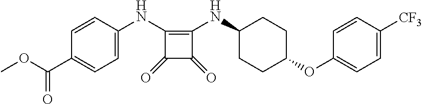 [SMILES: COC(=O)c1ccc(Nc2c(N[C@H]3CC[C@H](Oc4ccc(C(F)(F)F)cc4)CC3)c(=O)c2=O)cc1]

While stirring EtOH (1 mL) solution of methyl 4-((2-methoxy-3,4-dioxocyclobut-1-en-1-yl)amino)benzoate (40.0 mg, 0.15 mmol) at room temperature, (1,4-trans)-4-(4-(trifluoromethyl)phenoxy)cyclohexan-1-amine (59.5 mg, 0.23 mmol) was added under argon atmosphere, and the reaction mixture was stirred overnight. The resulting solids were filtered and washed with MeOH and EtOAc to obtain a yellow solid of Compound 58 (50 mg, 66%).

¹H NMR (DMSO-d₆, 600 MHz) δ 9.90 (s, 1H), 7.92 (d, 2H, J=8.8 Hz), 7.86 (m, 1H), 7.63 (d, 2H, J=8.7 Hz), 7.56 (d, 2H, J=8.2 Hz), 7.15 (d, 2H, J=8.6 Hz), 4.52 (m, 1H), 3.96 (m, 1H), 3.82 (s, 3H), 2.12 (m, 2H), 2.07 (m, 2H), 1.55 (m, 4H); ¹³C NMR (DMSO-d₆, 150 MHz) δ 184.3, 180.0, 169.1, 165.8, 163.0, 160.2, 143.4, 130.8, 127.3, 126.9, 125.5, 123.7, 123.0, 121.9, 121.2, 121.0, 120.8, 120.6, 117.4, 115.9, 74.0, 51.9, 51.9, 30.8, 29.2.

### 52. Methyl 4-((2-(((1,4-trans)-4-(4-cyanophenoxy)cyclohexyl)amino)-3,4-dioxocyclobut-1-en-1-yl)amino)benzoate (Compound 59)

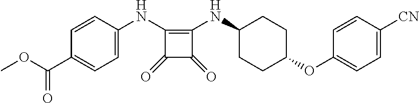 [SMILES: COC(=O)c1ccc(Nc2c(N[C@H]3CC[C@H](Oc4ccc(C#N)cc4)CC3)c(=O)c2=O)cc1]

While stirring EtOH (1 mL) solution of methyl 4-((2-methoxy-3,4-dioxocyclobut-1-en-1-yl)amino)benzoate (40.0 mg, 0.15 mmol) at room temperature, 4-(((1,4-trans)-4-aminocyclohexyl)oxy)benzonitrile (49.7 mg, 0.30 mmol) was added under argon atmosphere, and the reaction mixture was stirred overnight. The resulting solids were filtered and washed with MeOH and EtOAc to obtain a yellow solid of Compound 59 (53 mg, 77%).

¹H NMR (DMSO-d₆, 600 MHz) δ 9.85 (s, 1H), 7.92 (d, 2H, J=8.7 Hz), 7.82 (d, 1H, J=7.0 Hz), 7.75 (d, 2H, J=8.8 Hz), 7.55 (d, 2H, J=8.2 Hz), 7.15 (d, 2H, J=8.8 Hz), 4.55 (m, 1H), 3.95 (m, 1H), 3.82 (s, 3H), 2.09 (m, 4H), 1.55 (m, 4H); ¹³C NMR (DMSO-d₆, 150 MHz) δ 184.3, 180.0, 169.1, 165.8, 162.9, 160.9, 143.4, 134.2, 130.8, 123.0, 119.2, 117.4, 116.3, 102.6, 74.1, 51.9, 30.8, 29.1.

### 53. Methyl 4-((2-(((1,4-trans)-4-(4-nitrophenoxy)cyclohexyl)amino)-3,4-dioxocyclobut-1-en-1-yl)amino)benzoate (Compound 60)

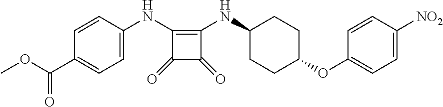 [SMILES: COC(=O)c1ccc(Nc2c(N[C@H]3CC[C@H](Oc4ccc([N+](=O)[O-])cc4)CC3)c(=O)c2=O)cc1]

While stirring EtOH (1 mL) solution of methyl 4-((2-methoxy-3,4-dioxocyclobut-1-en-1-yl)amino)benzoate (40.0 mg, 0.15 mmol) at room temperature, (1,4-trans)-4-(4-nitrophenoxy)cyclohexan-1-amine (54.2 mg, 0.23 mmol) was added under argon atmosphere, and the reaction mixture was stirred overnight. The resulting solids were filtered and washed with MeOH and EtOAc to obtain a yellow solid of Compound 60 (61 mg, 86%).

¹H NMR (DMSO-d₆, 500 MHz) δ 8.19 (d, 2H, J=9.1 Hz), 7.92 (d, 2H, J=8.5 Hz), 7.86 (m, 1H), 7.55 (d, 2H, J=7.6 Hz), 7.19 (d, 2H, J=9.1 Hz), 4.62 (m, 1H), 3.97 (m, 1H), 3.82 (s, 3H), 2.13 (m, 2H), 2.08 (m, 2H), 1.58 (m, 4H); ¹³C NMR (DMSO-d₆, 150 MHz) δ 184.3, 180.0, 169.1, 165.8, 163.0, 162.8, 143.4, 140.6, 130.8, 125.9, 123.0, 117.4, 115.7, 74.7, 51.9, 51.8, 30.7, 29.1.

### 54. Methyl 4-((2-(((1,4-trans)-4-(4-(methylsulfonyl)phenoxy)cyclohexyl)amino)-3,4-dioxocyclobut-1-en-1-yl)amino)benzoate (Compound 61)

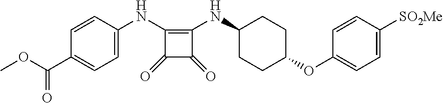 [SMILES: COC(=O)c1ccc(Nc2c(N[C@H]3CC[C@H](Oc4ccc(S(C)(=O)=O)cc4)CC3)c(=O)c2=O)cc1]

While stirring EtOH (1 mL) solution of methyl 4-((2-methoxy-3,4-dioxocyclobut-1-en-1-yl)amino)benzoate (40.0 mg, 0.15 mmol) at room temperature, (1,4-trans)-4-(4-(methylsulfonyl)phenoxy)cyclohexan-1-amine (62.0 mg, 0.23 mmol) was added under argon atmosphere, and the reaction mixture was stirred overnight. The resulting solids were filtered and washed with MeOH and EtOAc to obtain a yellow solid of Compound 61 (66 mg, 86%).

¹H NMR (DMSO-d₆, 500 MHz) δ 7.92 (d, 2H, J=8.4 Hz), 7.82 (m, 1H), 7.82 (d, 2H, J=8.6 Hz), 7.55 (d, 2H, J=7.5 Hz), 7.19 (d, 2H, J=8.4 Hz), 4.57 (m, 1H), 3.96 (m, 1H), 3.82 (s, 3H), 3.15 (s, 3H), 2.12 (m, 2H), 2.07 (m, 2H), 1.55 (m, 4H); ¹³C NMR (DMSO-d₆, 150 MHz) δ 184.3, 180.0, 169.1, 165.8, 163.0, 161.2, 143.4, 132.4, 130.8, 129.2, 123.0, 117.4, 115.8, 74.1, 51.9, 44.0, 43.9, 30.7, 29.1.

### 55. 3-((2-Fluorophenyl)amino)-4-(((1,4-trans)-4-(4-(trifluoromethyl)phenoxy)cyclohexyl)amino)cyclobut-3-ene-1,2-dione (Compound 62)

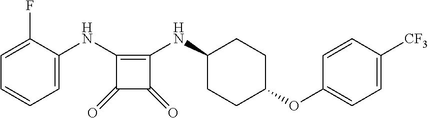 [SMILES: O=c1c(Nc2ccccc2F)c(N[C@H]2CC[C@H](Oc3ccc(C(F)(F)F)cc3)CC2)c1=O]

While stirring EtOH (1 mL) solution of 3-((2-fluorophenyl)amino)-4-methoxycyclobut-3-ene-1,2-dione (40.0 mg, 0.18 mmol) at room temperature, (1,4-trans)-4-(4-(trifluoromethyl)phenoxy)cyclohexan-1-amine (70.3 mg, 0.27 mmol) was added under argon atmosphere, and the reaction mixture was stirred overnight. The resulting solids were filtered and washed with MeOH and EtOAc to obtain a white solid of Compound 62 (44 mg, 55%).

¹H NMR (DMSO-d₆, 500 MHz) δ 9.45 (s, 1H), 8.16 (s, 1H), 7.99 (t, 1H, J=7.2 Hz), 7.63 (d, 2H, J=8.2 Hz), 7.27 (m, 1H), 7.18 (m, 1H), 7.16 (d, 2H, J=8.2 Hz), 7.05 (m, 1H), 4.54 (m, 1H), 3.97 (m, 1H), 2.10 (m, 4H), 1.55 (m, 4H); ¹³C NMR (DMSO-d₆, 125 MHz) δ 184.4, 179.9, 169.0, 163.1, 160.3, 153.1, 151.2, 127.9, 127.1, 127.1, 127.1, 127.0, 127.0, 125.7, 125.0, 125.0, 123.7, 123.6, 123.6, 121.4, 121.3, 121.1, 120.8, 120.5, 115.9, 115.7, 115.5, 74.0, 51.8, 30.9, 29.2.

### 56. 4-(((1,4-trans)-4-((2-((2-Fluorophenyl)amino)-3,4-dioxocyclobut-1-en-1-yl)amino)cyclohexyl)oxy)benzonitrile (Compound 63)

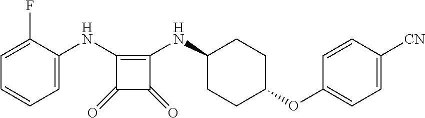 [SMILES: N#Cc1ccc(O[C@H]2CC[C@H](Nc3c(Nc4ccccc4F)c(=O)c3=O)CC2)cc1]

While stirring EtOH (1 mL) solution of 3-((2-fluorophenyl)amino)-4-methoxycyclobut-3-ene-1,2-dione (40.0 mg, 0.18 mmol) at room temperature, 4-(((1,4-trans)-4-aminocyclohexyl)oxy)benzonitrile (58.7 mg, 0.27 mmol) was added under argon atmosphere, and the reaction mixture was stirred overnight. The resulting solids were filtered and washed with MeOH and EtOAc to obtain a light brown solid of Compound 63 (44 mg, 60%).

¹H NMR (DMSO-d₆, 500 MHz) δ 8.19 (s, 1H), 7.98 (t, 1H, J=8.1 Hz), 7.75 (d, 1H, J=8.7 Hz), 7.27 (dd, 1H, J=11.4, 8.4 Hz), 7.17 (m, 1H), 7.15 (d, 2H, J=8.7 Hz), 7.05 (m, 1H), 4.56 (m, 1H), 3.97 (m, 1H), 2.09 (m, 4H), 1.55 (m, 4H); ¹³C NMR (DMSO-d₆, 125 MHz) δ 184.4, 180.0, 169.0, 163.2, 161.0, 153.2, 151.2, 134.3, 127.1, 127.0, 125.0, 123.7, 123.7, 121.1, 119.3, 116.4, 115.7, 115.5, 102.6, 74.1, 51.8, 30.9, 29.2.

### 57. 3-((2-Fluorophenyl)amino)-4-(((1,4-trans)-4-(4-nitrophenoxy)cyclohexyl)amino)cyclobut-3-ene-1,2-dione (Compound 64)

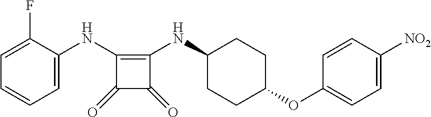 [SMILES: O=c1c(Nc2ccccc2F)c(N[C@H]2CC[C@H](Oc3ccc([N+](=O)[O-])cc3)CC2)c1=O]

While stirring EtOH (1 mL) solution of 3-((2-fluorophenyl)amino)-4-methoxycyclobut-3-ene-1,2-dione (40.0 mg, 0.18 mmol) at room temperature, (1,4-trans)-4-(4-nitrophenoxy)cyclohexan-1-amine (64.1 mg, 0.27 mmol) was added under argon atmosphere, and the reaction mixture was stirred overnight. The resulting solids were filtered and washed with MeOH and EtOAc to obtain a light yellow solid of Compound 64 (53 mg, 69%).

¹H NMR (DMSO-d₆, 500 MHz) δ 8.19 (d, 2H, J=8.8 Hz), 7.99 (m, 1H), 7.27 (m, 1H), 7.19 (d, 2H, J=8.7 Hz), 7.17 (m, 1H), 7.05 (m, 1H), 4.63 (m, 1H), 3.98 (m, 1H), 2.11 (m, 4H), 1.59 (m, 4H); ¹³C NMR (DMSO-d₆, 125 MHz) δ 184.3, 180.0, 169.0, 163.2, 163.0, 153.2, 151.2, 140.6, 127.1, 127.1, 126.0, 125.0, 125.0, 123.7, 123.6, 121.1, 115.8, 115.7, 115.5, 74.7, 51.7, 30.8, 29.2.

### 58. 3-((2-Fluorophenyl)amino)-4-(((1,4-trans)-4-(4-(methylsulfonyl)phenoxy)cyclohexyl)amino)cyclobut-3-ene-1,2-dione (Compound 65)

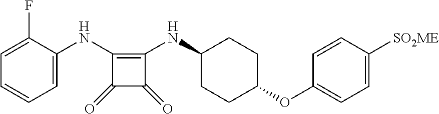

While stirring EtOH (1 mL) solution of 3-((2-fluorophenyl)amino)-4-methoxycyclobut-3-ene-1,2-dione (40.0 mg, 0.18 mmol) at room temperature, (1,4-trans)-4-(4-(methylsulfonyl)phenoxy)cyclohexan-1-amine (73.1 mg, 0.27 mmol) was added under argon atmosphere, and the reaction mixture was stirred overnight. The resulting solids were filtered and washed with MeOH and EtOAc to obtain a white solid of Compound 65 (20 mg, 24%).

¹H NMR (DMSO-d₆, 500 MHz) δ 8.17 (s, 1H), 7.98 (t, 1H, J=7.6 Hz), 7.83 (d, 2H, J=8.8 Hz), 7.27 (dd, 1H, J=11.1, 8.7 Hz), 7.20 (d, 2H, J=8.8 Hz), 7.18 (m, 1H), 7.05 (dd, 1H, J=12.3, 6.9 Hz), 4.58 (m, 1H), 3.98 (m, 1H), 3.16 (s, 3H), 2.10 (m, 4H), 1.56 (m, 4H); ¹³C NMR (DMSO-d₆, 125 MHz) δ 184.3, 179.9, 169.0, 163.2, 161.3, 153.1, 151.2, 132.4, 129.3, 127.1, 127.0, 125.0, 124.9, 123.7, 123.6, 121.1, 115.8, 115.6, 115.5, 74.2, 51.7, 44.0, 30.8, 29.1.

### 59. 3-((2-Chlorophenyl)amino)-4-(((1,4-trans)-4-(4-(trifluoromethyl)phenoxy)cyclohexyl)amino)cyclobut-3-ene-1,2-dione (Compound 66)

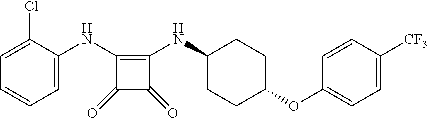 [SMILES: O=c1c(Nc2ccccc2Cl)c(N[C@H]2CC[C@H](Oc3ccc(C(F)(F)F)cc3)CC2)c1=O]

While stirring EtOH (1 mL) solution of 3-((2-chlorophenyl)amino)-4-methoxycyclobut-3-ene-1,2-dione (60.0 mg, 0.25 mmol) at room temperature, (1,4-trans)-4-(4-(trifluoromethyl)phenoxy)cyclohexan-1-amine (98.2 mg, 0.38 mmol) was added under argon atmosphere, and the reaction mixture was stirred overnight. The resulting solids were filtered and washed with MeOH and EtOAc to obtain a white solid of Compound 66 (99 mg, 84%).

¹H NMR (DMSO-d₆, 500 MHz) δ 9.18 (s, 1H), 8.30 (s, 1H), 7.66 (m, 1H), 7.63 (d, 2H, J=8.7 Hz), 7.47 (d, 1H, J=7.9 Hz), 7.33 (t, 1H, J=7.7 Hz), 7.15 (d, 2H, J=8.5 Hz), 7.09 (t, 1H, 7.5 Hz), 4.53 (m, 1H), 3.99 (m, 1H), 2.11 (m, 4H), 1.55 (m, 4H); ¹³C NMR (DMSO-d₆, 125 MHz) δ 184.9, 179.9, 169.0, 163.2, 160.3, 135.1, 129.6, 127.8, 127.0, 127.0, 127.0, 126.9, 125.7, 124.5, 123.5, 122.5, 122.3, 121.4, 121.3, 121.1, 120.8, 120.6, 115.9, 74.0, 51.9, 30.9, 29.3.

### 60. 4-(((1,4-trans)-4-((2-((2-Chlorophenyl)amino)-3,4-dioxocyclobut-1-en-1-yl)amino)cyclohexyl)oxy)benzonitrile (Compound 67)

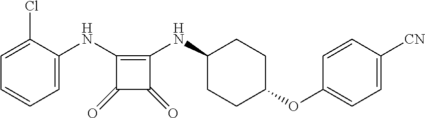 [SMILES: N#Cc1ccc(O[C@H]2CC[C@H](Nc3c(Nc4ccccc4Cl)c(=O)c3=O)CC2)cc1]

While stirring EtOH (1 mL) solution of 3-((2-chlorophenyl)amino)-4-methoxycyclobut-3-ene-1,2-dione (60.0 mg, 0.25 mmol) at room temperature, 4-(((1,4-trans)-4-aminocyclohexyl)oxy)benzonitrile (82.0 mg, 0.38 mmol) was added under argon atmosphere, and the reaction mixture was stirred overnight. The resulting solids were filtered and washed with MeOH and EtOAc to obtain an ivory solid of Compound 67 (97 mg, 91%).

¹H NMR (DMSO-d₆, 500 MHz) δ 9.21 (s, 1H), 8.32 (s, 1H), 7.75 (d, 2H, J=8.7 Hz), 7.64 (d, 1H, J=8.0 Hz), 7.48 (d, 1H, J=8.0 Hz), 7.33 (t, 1H, J=7.8 Hz), 7.15 (d, 2H, J=8.6 Hz), 7.09 (t, 1H, J=7.6 Hz), 4.55 (m, 1H), 3.98 (m, 1H), 2.10 (m, 4H), 1.55 (m, 4H); ¹³C NMR (DMSO-d₆, 125 MHz) δ 184.9, 179.9, 169.0, 163.2, 161.0, 135.1, 134.3, 129.6, 127.9, 124.5, 122.5, 122.3, 119.2, 116.4, 102.6, 74.2, 51.8, 30.9, 29.3.

### 61. 3-((2-Chlorophenyl)amino)-4-(((1,4-trans)-4-(4-nitrophenoxy)cyclohexyl)amino)cyclobut-3-ene-1,2-dione (Compound 68)

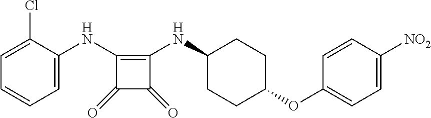 [SMILES: O=c1c(Nc2ccccc2Cl)c(N[C@H]2CC[C@H](Oc3ccc([N+](=O)[O-])cc3)CC2)c1=O]

While stirring EtOH (1 mL) solution of 3-((2-chlorophenyl)amino)-4-methoxycyclobut-3-ene-1,2-dione (60.0 mg, 0.25 mmol) at room temperature, (1,4-trans)-4-(4-nitrophenoxy)cyclohexan-1-amine (89.5 mg, 0.38 mmol) was added under argon atmosphere, and the reaction mixture was stirred overnight. The resulting solids were filtered and washed with MeOH and EtOAc to obtain a yellow solid of Compound 68 (100 mg, 90%).

¹H NMR (DMSO-d₆, 500 MHz) δ 8.32 (s, 1H), 8.19 (d, 2H, J=9.0 Hz), 7.64 (d, 1H, J=7.8 Hz), 7.48 (d, 1H, J=8.0 Hz), 7.33 (t, 1H, J=7.7 Hz), 7.19 (d, 2H, J=9.0 Hz), 7.09 (t, 1H, J=7.6 Hz), 4.62 (m, 1H), 3.99 (m, 1H), 2.12 (m, 4H), 1.57 (m, 4H); ¹³C NMR (DMSO-d₆, 125 MHz) δ 184.9, 179.9, 169.1, 163.2, 162.9, 140.6, 135.2, 129.6, 127.9, 126.0, 124.5, 122.5, 122.3, 115.8, 74.8, 51.8, 30.9, 29.3.

### 62. 3-((2-Chlorophenyl)amino)-4-(((1,4-trans)-4-(4-(methylsulfonyl)phenoxy)cyclohexyl)amino)cyclobut-3-ene-1,2-dione (Compound 69)

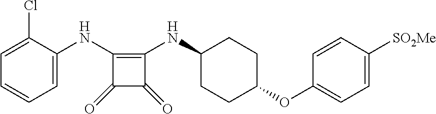 [SMILES: CS(=O)(=O)c1ccc(O[C@H]2CC[C@H](Nc3c(Nc4ccccc4Cl)c(=O)c3=O)CC2)cc1]

While stirring EtOH (1 mL) solution of 3-((2-chlorophenyl)amino)-4-methoxycyclobut-3-ene-1,2-dione (60.0 mg, 0.25 mmol) at room temperature, (1,4-trans)-4-(4-(methylsulfonyl)phenoxy)cyclohexan-1-amine (102.0 mg, 0.38 mmol) was added under argon atmosphere, and the reaction mixture was stirred overnight. The resulting solids were filtered and washed with MeOH and EtOAc to obtain an ivory solid of Compound 69 (48 mg, 40%).

¹H NMR (DMSO-d₆, 500 MHz) δ 9.24 (s, 1H), 8.32 (d, 1H, J=7.9 Hz), 7.83 (d, 2H, J=8.9 Hz), 7.63 (d, 1H, J=8.1 Hz), 7.48 (d, 1H, J=7.8 Hz), 7.33 (t, 1H, J=7.7 Hz), 7.20 (d, 2H, J=8.9 Hz), 7.09 (t, 1H, J=7.7 Hz), 4.58 (m, 1H), 3.99 (m, 1H), 3.16 (s, 3H), 2.11 (m, 4H), 1.56 (m, 4H); ¹³C NMR (DMSO-d₆, 100 MHz) δ 184.9, 179.9, 169.1, 163.2, 161.3, 135.1, 132.4, 129.6, 129.3, 127.9, 124.6, 122.5, 122.3, 115.8, 74.3, 51.9, 44.0, 30.9, 29.3.

### 63. 3-(((1,4-trans)-4-(4-(Trifluoromethyl)phenoxy)cyclohexyl)amino)-4-((2-(trifluoromethyl)phenyl)amino)cyclobut-3-ene-1,2-dione (Compound 70)

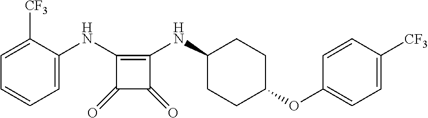 [SMILES: O=c1c(Nc2ccccc2C(F)(F)F)c(N[C@H]2CC[C@H](Oc3ccc(C(F)(F)F)cc3)CC2)c1=O]

While stirring EtOH (1 mL) solution of 3-methoxy-4-((2-(trifluoromethyl)phenyl)amino)cyclobut-3-ene-1,2-dione (60.0 mg, 0.22 mmol) at room temperature, (1,4-trans)-4-(4-(trifluoromethyl)phenoxy)cyclohexan-1-amine (86.1 mg, 0.33 mmol) was added under argon atmosphere, and the reaction mixture was stirred overnight. The resulting solids were filtered and washed with MeOH and EtOAc to obtain a white solid of Compound 70 (62 mg, 56%).

¹H NMR (CDCl₃, 500 MHz) δ 8.49 (s, 1H), 8.25 (s, 1H), 7.51 (d, 2H, J=8.5 Hz), 7.39 (d, 1H, J=7.8 Hz), 7.32 (t, 1H, J=7.6 Hz), 7.24 (d, 1H, J=7.9 Hz), 6.99 (t, 1H, J=7.4 Hz), 6.92 (d, 1H, J=8.5 Hz), 4.27 (m, 1H), 4.11 (m, 1H), 2.09 (m, 4H), 1.53 (m, 4H); ¹³C NMR (CDCl₃, 125 MHz) δ 183.3, 181.4, 170.8, 162.8, 160.1, 134.9, 132.3, 127.8, 127.1, 127.1, 127.0, 127.0, 126.9, 126.3, 126.3, 126.2, 125.6, 125.0, 124.9, 124.7, 123.4, 123.3, 123.1, 122.8, 122.5, 122.5, 121.8, 121.6, 121.3, 121.3, 121.1, 120.3, 115.7, 74.2, 52.9, 31.1, 29.4.

### 64. 4-(((1,4-trans)-4-((3,4-Dioxo-2-((2-(trifluoromethyl)phenyl)amino)cyclobut-1-en-1-yl)amino)cyclohexyl)oxy)benzonitrile (Compound 71)

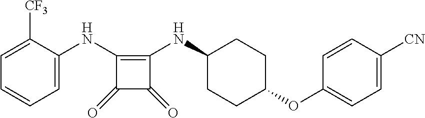 [SMILES: N#Cc1ccc(O[C@H]2CC[C@H](Nc3c(Nc4ccccc4C(F)(F)F)c(=O)c3=O)CC2)cc1]

While stirring EtOH (1 mL) solution of 3-methoxy-4-((2-(trifluoromethyl)phenyl)amino)cyclobut-3-ene-1,2-dione (60.0 mg, 0.22 mmol) at room temperature, 4-(((1,4-trans)-4-aminocyclohexyl)oxy)benzonitrile (71.8 mg, 0.33 mmol) was added under argon atmosphere, and the reaction mixture was stirred overnight. The resulting solids were filtered and washed with MeOH and EtOAc to obtain an ivory solid of Compound 71 (79 mg, 78%).

¹H NMR (CDCl₃, 500 MHz) δ 8.33 (s, 1H), 7.55 (d, 2H, J=8.3 Hz), 7.38 (d, 1H, J=7.3 Hz), 7.31 (t, 1H, J=6.9 Hz), 7.25 (m, 1H), 6.98 (t, 1H, J=6.8 Hz), 6.91 (d, 2H, J=8.3 Hz), 4.29 (m, 1H), 4.11 (m, 1H), 2.08 (m, 4H), 1.54 (m, 4H); ¹³C NMR (CDCl₃, 125 MHz) δ 183.3, 181.3, 170.8, 162.8, 161.1, 134.9, 134.2, 132.3, 126.8, 126.2, 126.2, 125.0, 124.9, 124.7, 122.5, 121.7, 121.5, 121.2, 121.0, 120.3, 119.4, 116.2, 103.7, 74.4, 52.7, 31.0, 29.4.

### 65. 3-(((1,4-trans)-4-(4-Nitrophenoxy)cyclohexyl)amino)-4-((2-(trifluoromethyl)phenyl)amino)cyclobut-3-ene-1,2-dione (Compound 72)

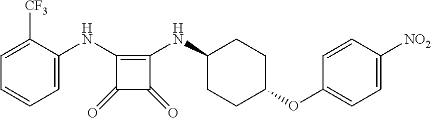 [SMILES: O=c1c(Nc2ccccc2C(F)(F)F)c(N[C@H]2CC[C@H](Oc3ccc([N+](=O)[O-])cc3)CC2)c1=O]

While stirring EtOH (1 mL) solution of 3-methoxy-4-((2-(trifluoromethyl)phenyl)amino)cyclobut-3-ene-1,2-dione (60.0 mg, 0.22 mmol) at room temperature, (1,4-trans)-4-(4-nitrophenoxy)cyclohexan-1-amine (78.4 mg, 0.33 mmol) was added under argon atmosphere, and the reaction mixture was stirred overnight. The resulting solids were filtered and washed with MeOH and EtOAc to obtain a light yellow solid of Compound 72 (55 mg, 52%).

¹H NMR (DMSO-d₆, 500 MHz) δ 9.00 (s, 1H), 8.20 (m, 1H), 8.19 (d, 2H, J=8.9 Hz), 7.71 (d, 1H, J=7.6 Hz), 7.66 (t, 1H, J=7.5 Hz), 7.39 (d, 1H, J=7.6 Hz), 7.33 (t, 1H, J=7.1 Hz), 7.19 (d, 2H, J=9.0 Hz), 4.62 (m, 1H), 3.99 (m, 1H), 2.12 (m, 4H), 1.58 (m, 4H); ¹³C NMR (DMSO-d₆, 125 MHz) δ 185.5, 180.1, 169.2, 163.9, 162.9, 140.6, 135.2, 133.3, 127.2, 126.2, 126.2, 126.0, 125.7, 125.1, 124.6, 122.9, 120.7, 119.9, 119.7, 119.5, 119.2, 115.8, 74.9, 51.8, 31.0, 29.4.

### 66. 3-(((1,4-trans)-4-(4-(Methylsulfonyl)phenoxy)cyclohexyl)amino)-4-((2-(trifluoromethyl)phenyl)amino)cyclobut-3-ene-1,2-dione (Compound 73)

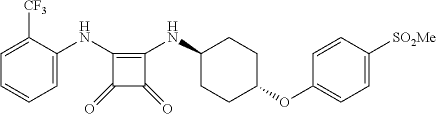 [SMILES: CS(=O)(=O)c1ccc(O[C@H]2CC[C@H](Nc3c(Nc4ccccc4C(F)(F)F)c(=O)c3=O)CC2)cc1]

While stirring EtOH (1 mL) solution of 3-methoxy-4-((2-(trifluoromethyl)phenyl)amino)cyclobut-3-ene-1,2-dione (60.0 mg, 0.22 mmol) at room temperature, (1,4-trans)-4-(4-(methylsulfonyl)phenoxy)cyclohexan-1-amine (89.4 mg, 0.33 mmol) was added under argon atmosphere, and the reaction mixture was stirred overnight. The resulting solids were filtered and washed with MeOH and EtOAc to obtain an ivory solid of Compound 73 (34 mg, 31%).

¹H NMR (CDCl₃, 500 MHz) δ 7.93 (s, 1H), 7.83 (d, 2H, J=8.6 Hz), 7.43 (d, 1H, J=7.8 Hz), 7.38 (t, 1H, J=7.6 Hz), 7.28 (d, 1H, J=8.5 Hz), 7.04 (t, 1H, J=7.4 Hz), 6.98 (d, 2H, J=8.7 Hz), 4.28 (m, 1H), 4.11 (m, 1H), 3.03 (s, 3H), 2.09 (m, 4H), 1.52 (m, 4H); ¹³C NMR (CDCl₃, 125 MHz) δ 185.4, 180.1, 169.1, 163.9, 161.3, 135.1, 133.2, 132.4, 129.3, 127.2, 126.3, 126.2, 126.2, 126.1, 125.7, 125.0, 124.6, 122.9, 120.7, 120.0, 119.7, 119.5, 119.3, 115.8, 74.3, 51.8, 44.0, 31.0, 29.4.

### 67. 3-((2-(tert-Butyl)phenyl)amino)-4-(((1,4-trans)-4-(4-(trifluoromethyl)phenoxy)cyclohexyl)amino)cyclobut-3-ene-1,2-dione (Compound 74)

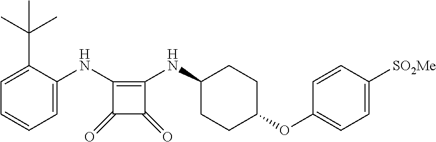 [SMILES: CC(C)(C)c1ccccc1Nc1c(N[C@H]2CC[C@H](Oc3ccc(S(C)(=O)=O)cc3)CC2)c(=O)c1=O]

While stirring EtOH (1 mL) solution of 3-((2-(tert-butyl)phenyl)amino)-4-methoxycyclobut-3-ene-1,2-dione (50.0 mg, 0.19 mmol) at room temperature, (1,4-trans)-4-(4-(trifluoromethyl)phenoxy)cyclohexan-1-amine (75.0 mg, 0.29 mmol) was added under argon atmosphere, and the reaction mixture was stirred overnight. The resulting solids were filtered and washed with MeOH and EtOAc to obtain an ivory solid of Compound 74 (63 mg, 67%).

¹H NMR (CDCl₃, 500 MHz) δ 7.94 (s, 1H), 7.50 (d, 2H, J=8.6 Hz), 7.39 (d, 1H, J=7.4 Hz), 7.13 (m, 2H), 6.97 (d, 1H, J=7.3 Hz), 6.90 (d, 2H, J=8.6 Hz), 4.22 (m, 1H), 4.02 (m, 1H), 2.03 (m, 4H), 1.50 (m, 2H), 1.40 (m, 2H), 1.31 (s, 9H); ¹³C NMR (CDCl₃, 100 MHz) δ 183.6, 183.2, 168.5, 165.1, 160.2, 145.4, 135.5, 128.6, 127.8, 127.8, 127.6, 127.1, 127.1, 127.0, 127.0, 126.8, 125.9, 123.6, 123.3, 123.2, 122.9, 122.6, 120.5, 115.8, 74.4, 52.5, 35.3, 31.4, 30.8, 29.5.

### 68. 4-(((1,4-trans)-4-((2-((2-(tert-Butyl)phenyl)amino)-3,4-dioxocyclobut-1-en-1-yl)amino)cyclohexyl)oxy)benzonitrile (Compound 75)

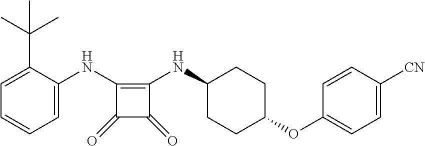 [SMILES: CC(C)(C)c1ccccc1Nc1c(N[C@H]2CC[C@H](Oc3ccc(C#N)cc3)CC2)c(=O)c1=O]

While stirring EtOH (1 mL) solution of 3-((2-(tert-butyl)phenyl)amino)-4-methoxycyclobut-3-ene-1,2-dione (60.0 mg, 0.23 mmol) at room temperature, 4-(((1,4-trans)-4-aminocyclohexyl)oxy)benzonitrile (75.0 mg, 0.34 mmol) was added under argon atmosphere, and the reaction mixture was stirred overnight. The resulting solids were filtered and washed with MeOH and EtOAc to obtain an ivory solid of Compound 75 (61 mg, 59%).

¹H NMR (CDCl₃, 500 MHz) δ 7.54 (d, 2H, J=8.4 Hz), 7.39 (d, 1H, J=7.3 Hz), 7.14 (m, 2H), 6.97 (d, 1H, J=7.4 Hz), 6.89 (d, 2H, J=8.5 Hz), 4.24 (m, 1H), 4.02 (m, 1H), 2.03 (m, 4H), 1.51 (m, 2H), 1.41 (m, 2H), 1.32 (s, 9H); ¹³C NMR (CDCl₃, 125 MHz) δ 183.3, 182.8, 168.6, 165.0, 161.1, 145.1, 135.4, 134.2, 127.8, 127.7, 127.4, 126.7, 119.4, 116.2, 103.9, 74.5, 52.4, 35.2, 31.3, 30.8, 29.5.

### 69. 3-((2-(tert-Butyl)phenyl)amino)-4-(((1,4-trans)-4-(4-nitrophenoxy)cyclohexyl)amino)cyclobut-3-ene-1,2-dione (Compound 76)

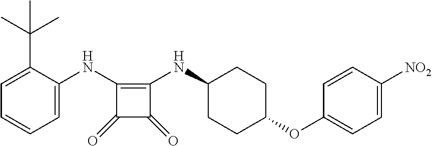 [SMILES: CC(C)(C)c1ccccc1Nc1c(N[C@H]2CC[C@H](Oc3ccc([N+](=O)[O-])cc3)CC2)c(=O)c1=O]

While stirring EtOH (1 mL) solution of 3-((2-(tert-butyl)phenyl)amino)-4-methoxycyclobut-3-ene-1,2-dione (50.0 mg, 0.19 mmol) at room temperature, (1,4-trans)-4-(4-nitrophenoxy)cyclohexan-1-amine (68.0 mg, 0.28 mmol) was added under argon atmosphere, and the reaction mixture was stirred overnight. The resulting solids were filtered and washed with MeOH and EtOAc to obtain a light brown solid of Compound 76 (29 mg, 32%).

¹H NMR (DMSO-d₆, 500 MHz) δ 8.19 (d, 2H, J=9.2 Hz), 7.76 (s, 1H), 7.40 (m, 1H), 7.22 (m, 2H), 7.19 (d, 2H, J=9.2 Hz), 7.08 (m, 1H), 4.60 (m, 1H), 3.95 (m, 1H), 2.11 (m, 4H), 1.57 (m, 4H), 1.37 (s, 9H); ¹³C NMR (DMSO-d₆, 125 MHz) δ 184.2, 180.8, 168.3, 166.0, 162.8, 143.8, 140.6, 135.9, 128.8, 126.4, 126.4, 125.9, 115.7, 115.4, 74.9, 51.5, 34.7, 30.9, 30.6, 29.3.

### 70. 3-((2-(tert-Butyl)phenyl)amino)-4-(((1,4-trans)-4-(4-(methylsulfonyl)phenoxy)cyclohexyl)amino)cyclobut-3-ene-1,2-dione (Compound 77)

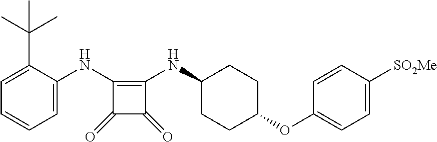 [SMILES: CC(C)(C)c1ccccc1Nc1c(N[C@H]2CC[C@H](Oc3ccc(S(C)(=O)=O)cc3)CC2)c(=O)c1=O]

While stirring EtOH (1 mL) solution of 3-((2-(tert-butyl)phenyl)amino)-4-methoxycyclobut-3-ene-1,2-dione (40.0 mg, 0.15 mmol) at room temperature, (1,4-trans)-4-(4-(methylsulfonyl)phenoxy)cyclohexan-1-amine (62.3 mg, 0.23 mmol) was added under argon atmosphere, and the reaction mixture was stirred overnight. The resulting solids were filtered and washed with MeOH and EtOAc to obtain a white solid of Compound 77 (18 mg, 23%).

¹H NMR (CDCl₃, 500 MHz) δ 7.82 (d, 2H, J=8.7 Hz), 7.43 (d, 1H, J=7.3 Hz), 7.21 (m, 2H), 6.98 (m, 1H), 6.95 (d, 2H, J=8.7 Hz), 4.26 (m, 1H), 4.03 (m, 1H), 3.02 (s, 3H), 2.04 (m, 4H), 1.54 (m, 2H), 1.37 (m, 2H), 1.36 (s, 9H); ¹³C NMR (CDCl₃, 125 MHz) δ 183.3, 182.7, 168.6, 165.0, 161.9, 145.1, 135.4, 132.1, 129.7, 127.9, 127.7, 127.4, 126.7, 115.9, 74.6, 52.4, 45.0, 35.1, 31.3, 30.8, 29.4.

### 71. 3-((2-Methoxyphenyl)amino)-4-(((1,4-trans)-4-(4-(trifluoromethyl)phenoxy)cyclohexyl)amino)cyclobut-3-ene-1,2-dione (Compound 78)

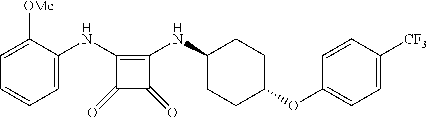 [SMILES: COc1ccccc1Nc1c(N[C@H]2CC[C@H](Oc3ccc(C(F)(F)F)cc3)CC2)c(=O)c1=O]

While stirring EtOH (1 mL) solution of 3-methoxy-4-((2-methoxyphenyl)amino)cyclobut-3-ene-1,2-dione (40.0 mg, 0.15 mmol) at room temperature, (1,4-trans)-4-(4-(trifluoromethyl)phenoxy)cyclohexan-1-amine (66.7 mg, 0.26 mmol) was added under argon atmosphere, and the reaction mixture was stirred overnight. The resulting solids were filtered and washed with MeOH and EtOAc to obtain a white solid of Compound 78 (71 mg, 90%).

¹H NMR (DMSO-d₆, 500 MHz) δ 9.18 (s, 1H), 8.31 (s, 1H), 7.77 (d, 1H, J=7.6 Hz), 7.63 (d, 2H, J=8.2 Hz), 7.16 (d, 2H, J=8.2 Hz), 7.06 (d, 1H, J=7.8 Hz), 7.03 (m, 1H), 6.93 (t, 1H, J=7.3 Hz), 4.53 (m, 1H), 3.98 (m, 1H), 3.91 (s, 3H), 2.10 (m, 4H), 1.54 (m, 4H); ¹³C NMR (DMSO-d₆, 125 MHz) δ 184.0, 180.0, 168.7, 163.5, 160.3, 148.6, 127.9, 127.7, 127.1, 127.0, 127.0, 127.0, 125.7, 123.7, 123.6, 121.4, 121.3, 121.1, 120.8, 120.7, 120.5, 120.0, 115.9, 111.3, 74.0, 55.9, 51.8, 30.9, 29.3.

### 72. 4-(((1,4-trans)-4-((2-((2-Methoxyphenyl)amino)-3,4-dioxocyclobut-1-en-1-yl)amino)cyclohexyl)oxy)benzonitrile (Compound 79)

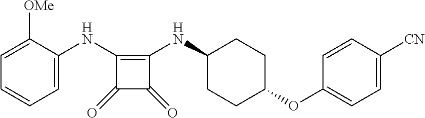 [SMILES: COc1ccccc1Nc1c(N[C@H]2CC[C@H](Oc3ccc(C#N)cc3)CC2)c(=O)c1=O]

While stirring EtOH (1 mL) solution of 3-methoxy-4-((2-methoxyphenyl)amino)cyclobut-3-ene-1,2-dione (40.0 mg, 0.17 mmol) at room temperature, 4-(((1,4-trans)-4-aminocyclohexyl)oxy)benzonitrile (55.6 mg, 0.26 mmol) was added under argon atmosphere, and the reaction mixture was stirred overnight. The resulting solids were filtered and washed with MeOH and EtOAc to obtain an ivory solid of Compound 79 (61 mg, 85%).

¹H NMR (DMSO-d₆, 500 MHz) δ 9.18 (s, 1H), 8.32 (d, 1H, J=6.2 Hz), 7.76 (m, 1H), 7.75 (d, 2H, J=8.6 Hz), 7.15 (d, 2H, J=8.7 Hz), 7.05 (m, 1H), 7.03 (m, 1H), 6.92 (m, 1H), 4.55 (m, 1H), 3.97 (m, 1H), 3.90 (s, 3H), 2.09 (m, 4H), 1.54 (m, 4H); ¹³C NMR (DMSO-d₆, 125 MHz) δ 184.1, 180.0, 168.7, 163.6, 161.0, 148.6, 134.3, 127.7, 123.7, 120.8, 120.0, 119.3, 116.4, 111.3, 102.6, 74.2, 55.9, 51.7, 30.9, 29.2.

### 73. 3-((2-Methoxyphenyl)amino)-4-(((1,4-trans)-4-(4-nitrophenoxy)cyclohexyl)amino)cyclobut-3-ene-1,2-dione (Compound 80)

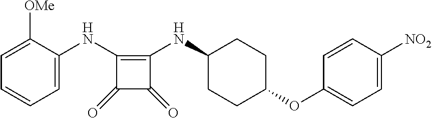 [SMILES: COc1ccccc1Nc1c(N[C@H]2CC[C@H](Oc3ccc([N+](=O)[O-])cc3)CC2)c(=O)c1=O]

While stirring EtOH (1 mL) solution of 3-methoxy-4-((2-methoxyphenyl)amino)cyclobut-3-ene-1,2-dione (40.0 mg, 0.17 mmol) at room temperature, (1,4-trans)-4-(4-nitrophenoxy)cyclohexan-1-amine (60.8 mg, 0.26 mmol) was added under argon atmosphere, and the reaction mixture was stirred overnight. The resulting solids were filtered and washed with MeOH and EtOAc to obtain a light yellow solid of Compound 80 (61 mg, 81%).

¹H NMR (DMSO-d₆, 500 MHz) δ 9.18 (s, 1H), 8.33 (s, 1H), 8.19 (d, 2H, J=9.1 Hz), 7.77 (d, 1H, J=7.7 Hz), 7.19 (d, 2H, J=9.1 Hz), 7.06 (d, 1H, J=8.1 Hz), 7.03 (m, 1H), 6.92 (t, 1H, J=7.5 Hz), 4.62 (m, 1H), 3.99 (m, 1H), 3.91 (s, 3H), 2.13 (m, 2H), 2.08 (m, 2H), 1.57 (m, 4H); ¹³C NMR (DMSO-d₆, 125 MHz) δ 184.0, 180.0, 168.7, 163.5, 162.9, 148.6, 140.6, 127.7, 126.0, 123.7, 120.7, 119.9, 115.8, 111.3, 74.8, 55.9, 51.7, 30.9, 29.2.

### 74. 3-((2-Methoxyphenyl)amino)-4-(((1,4-trans)-4-(4-(methylsulfonyl)phenoxy)cyclohexyl)amino)cyclobut-3-ene-1,2-dione (Compound 81)

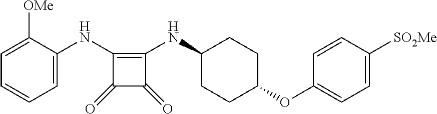 [SMILES: COc1ccccc1Nc1c(N[C@H]2CC[C@H](Oc3ccc(S(C)(=O)=O)cc3)CC2)c(=O)c1=O]

While stirring EtOH (1 mL) solution of 3-methoxy-4-((2-methoxyphenyl)amino)cyclobut-3-ene-1,2-dione (40.0 mg, 0.17 mmol) at room temperature, (1,4-trans)-4-(4-(methylsulfonyl)phenoxy)cyclohexan-1-amine (69.3 mg, 0.26 mmol) was added under argon atmosphere, and the reaction mixture was stirred overnight. The resulting solids were filtered and washed with MeOH and EtOAc to obtain an ivory solid of Compound 81 (72 mg, 89%).

¹H NMR (DMSO-d₆, 400 MHz) δ 8.34 (d, 1H, J=6.2 Hz), 7.83 (d, 2H, J=8.9 Hz), 7.77 (d, 1H, J=7.9 Hz), 7.20 (d, 2H, J=8.9 Hz), 7.05 (m, 1H), 7.04 (m, 1H), 6.92 (m, 1H), 4.57 (m, 1H), 3.98 (m, 1H), 3.90 (s, 3H), 3.16 (s, 3H), 2.10 (m, 4H), 1.55 (m, 4H); ¹³C NMR (DMSO-d₆, 100 MHz) δ 184.0, 180.0, 168.7, 163.5, 161.3, 148.6, 132.4, 129.3, 127.7, 123.7, 120.7, 119.9, 115.8, 111.3, 74.2, 55.9, 51.7, 44.0, 30.8, 29.2.

### 75. 4-(((1,4-trans)-4-((2-((2-Methoxyphenyl)amino)-3,4-dioxocyclobut-1-en-1-yl)amino)cyclohexyl)oxy)benzenesulfonamide (Compound 82)

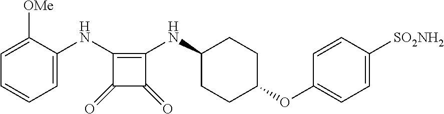 [SMILES: COc1ccccc1Nc1c(N[C@H]2CC[C@H](Oc3ccc(S(N)(=O)=O)cc3)CC2)c(=O)c1=O]

While stirring EtOH (1 mL) solution of 3-methoxy-4-((2-methoxyphenyl)amino)cyclobut-3-ene-1,2-dione (40.0 mg, 0.17 mmol) at room temperature, 4-(((1,4-trans)-4-aminocyclohexyl)oxy)benzenesulfonamide (69.3 mg, 0.26 mmol) was added under argon atmosphere, and the reaction mixture was stirred overnight. The resulting solids were filtered and washed with MeOH and EtOAc to obtain an ivory solid of Compound 82 (72 mg, 89%).

¹H NMR (DMSO-d₆, 500 MHz) δ 9.18 (s, 1H), 8.31 (d, 1H, J=7.7 Hz), 7.77 (d, 1H, J=8.0 Hz), 7.74 (d, 2H, J=8.8 Hz), 7.20 (s, 2H), 7.13 (d, 2H, J=8.9 Hz), 7.06 (dd, 1H, J=8.3, 1.5 Hz), 7.03 (m, 1H), 6.93 (m, 1H), 4.52 (m, 1H), 3.98 (m. 1H), 3.91 (s, 3H), 2.10 (m, 4H), 1.54 (m, 4H); ¹³C NMR (DMSO-d₆, 125 MHz) δ 184.0, 180.0, 168.7, 163.5, 159.8, 148.6, 136.0, 127.7, 127.7, 123.7, 120.7, 119.9, 115.4, 111.3, 74.0, 55.9, 51.7, 30.9, 29.2.

### 76. 3-((3,4-Dioxo-2-(((1,4-trans)-4-(4-(trifluoromethyl)phenoxy)cyclohexyl)amino)cyclobut-1-en-1-yl)amino)-5-(trifluoromethyl)benzonitrile (Compound 83)

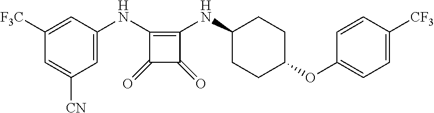 [SMILES: N#Cc1cc(Nc2c(N[C@H]3CC[C@H](Oc4ccc(C(F)(F)F)cc4)CC3)c(=O)c2=O)cc(C(F)(F)F)c1]

While stirring EtOH (1 mL) solution of 3-((2-methoxy-3,4-dioxocyclobut-1-en-1-yl)amino)-5-(trifluoromethyl)benzonitrile (50.0 mg, 0.17 mmol) at room temperature, (1,4-trans)-4-(4-(trifluoromethyl)phenoxy)cyclohexan-1-amine (66.0 mg, 0.25 mmol) was added under argon atmosphere, and the reaction mixture was stirred overnight. The resulting solids were filtered and washed with MeOH and EtOAc to obtain a light yellow solid of Compound 83 (56 mg, 64%).

¹H NMR (DMSO-d₆, 500 MHz) δ 10.09 (s, 1H), 8.08 (s, 1H), 8.02 (s, 1H), 7.91 (s, 1H), 7.89 (s, 1H), 7.62 (d, 2H, J=8.6 Hz), 7.15 (d, 2H, J=8.6 Hz), 4.52 (m, 1H), 3.97 (m. 1H), 2.12 (m, 2H), 2.06 (m, 2H), 1.55 (m, 4H); ¹³C NMR (DMSO-d₆, 125 MHz) δ 184.5, 180.4, 169.2, 162.5, 160.3, 140.9, 131.8, 131.5, 131.3, 131.0, 127.9, 127.1, 127.0, 127.0, 127.0, 126.3, 125.7, 124.6, 124.1, 123.6, 122.0, 122.0, 121.9, 121.4, 121.4, 121.1, 120.9, 120.6, 119.8, 118.9, 117.5, 115.9, 113.6. 74.0, 52.1, 30.8, 29.2.

### 77. 3-((2-(((1,4-trans)-4-(4-Cyanophenoxy)cyclohexyl)amino)-3,4-dioxocyclobut-1-en-1-yl)amino)-5-(trifluoromethyl)benzonitrile (Compound 84)

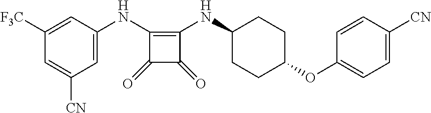 [SMILES: N#Cc1ccc(O[C@H]2CC[C@H](Nc3c(Nc4cc(C#N)cc(C(F)(F)F)c4)c(=O)c3=O)CC2)cc1]

While stirring EtOH (1 mL) solution of 3-((2-methoxy-3,4-dioxocyclobut-1-en-1-yl)amino)-5-(trifluoromethyl)benzonitrile (50.0 mg, 0.17 mmol) at room temperature, 4-(((1,4-trans)-4-aminocyclohexyl)oxy)benzonitrile (55.0 mg, 0.25 mmol) was added under argon atmosphere, and the reaction mixture was stirred overnight. The resulting solids were filtered and washed with MeOH and EtOAc to obtain a light yellow solid of Compound 84 (56 mg, 68%).

¹H NMR (DMSO-d₆, 500 MHz) δ 8.07 (s, 1H), 8.01 (s, 1H), 7.88 (s, 1H), 7.74 (d, 2H, J=8.7 Hz), 7.14 (d, 2H, J=8.8 Hz), 4.54 (m, 1H), 3.96 (m, 1H), 2.10 (m, 2H), 2.05 (m, 2H), 1.55 (m, 4H); ¹³C NMR (DMSO-d₆, 125 MHz) δ 184.5, 180.4, 169.2, 162.5, 161.0, 140.9, 134.3, 131.8, 131.6, 131.3, 131.0, 126.3, 124.7, 124.1, 122.0, 122.0, 121.9, 119.8, 119.3, 118.9, 118.9, 117.5, 116.4, 113.6, 102.6, 74.2, 52.1, 30.8, 29.2.

### 78. 3-((2-(((1,4-trans)-4-(4-Nitrophenoxy)cyclohexyl)amino)-3,4-dioxocyclobut-1-en-1-yl)amino)-5-(trifluoromethyl)benzonitrile (Compound 85)

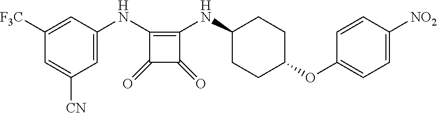 [SMILES: N#Cc1cc(Nc2c(N[C@H]3CC[C@H](Oc4ccc([N+](=O)[O-])cc4)CC3)c(=O)c2=O)cc(C(F)(F)F)c1]

While stirring EtOH (1 mL) solution of 3-((2-methoxy-3,4-dioxocyclobut-1-en-1-yl)amino)-5-(trifluoromethyl)benzonitrile (50.0 mg, 0.17 mmol) at room temperature, (1,4-trans)-4-(4-nitrophenoxy)cyclohexan-1-amine (60.0 mg, 0.25 mmol) was added under argon atmosphere, and the reaction mixture was stirred overnight. The resulting solids were filtered and washed with MeOH and EtOAc to obtain a brown solid of Compound 85 (49 mg, 57%).

¹H NMR (DMSO-d₆, 500 MHz) δ 10.07 (s, 1H), 8.18 (d, 2H, J=9.2 Hz), 8.08 (s, 1H), 8.01 (s, 1H), 7.89 (s, 1H), 7.18 (d, 2H, J=9.2 Hz), 4.61 (m, 1H), 3.98 (m, 1H), 2.13 (m, 2H), 2.07 (m, 2H), 1.58 (m, 4H); ¹³C NMR (DMSO-d₆, 125 MHz) δ 184.5, 180.4, 169.1, 162.9, 162.5, 140.9, 140.6, 131.8, 131.5, 131.3, 131.0, 126.3, 126.0, 124.7, 124.1, 122.0, 122.0, 121.9, 119.8, 118.9, 118.9, 118.9, 117.5, 115.8, 113.6, 74.7, 52.0, 30.7, 29.2.

### 79. 3-((2-(((1,4-trans)-4-(4-(Methylsulfonyl)phenoxy)cyclohexyl)amino)-3,4-dioxocyclobut-1-en-1-yl)amino)-5-(trifluoromethyl)benzonitrile (Compound 86)

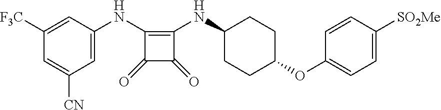 [SMILES: CS(=O)(=O)c1ccc(O[C@H]2CC[C@H](Nc3c(Nc4cc(C#N)cc(C(F)(F)F)c4)c(=O)c3=O)CC2)cc1]

While stirring EtOH (1 mL) solution of 3-((2-methoxy-3,4-dioxocyclobut-1-en-1-yl)amino)-5-(trifluoromethyl)benzonitrile (50.0 mg, 0.17 mmol) at room temperature, (1,4-trans)-4-(4-(methylsulfonyl)phenoxy)cyclohexan-1-amine (68.0 mg, 0.25 mmol) was added under argon atmosphere, and the reaction mixture was stirred overnight. The resulting solids were filtered and washed with MeOH and EtOAc to obtain a light yellow solid of Compound 86 (61 mg, 68%).

¹H NMR (DMSO-d₆, 500 MHz) δ 8.08 (s, 1H), 8.02 (s, 1H), 7.89 (s, 1H), 7.82 (d, 2H, J=8.8 Hz), 7.20 (d, 2H, J=8.9 Hz), 4.56 (m, 1H), 3.98 (m, 1H), 3.15 (s, 3H), 2.13 (m, 2H), 2.06 (m, 2H), 1.56 (m, 4H); ¹³C NMR (DMSO-d₆, 125 MHz) δ 184.5, 180.4, 169.2, 162.5, 161.3, 140.9, 132.4, 131.8, 131.5, 131.3, 131.0, 129.3, 126.3, 124.7, 124.1, 122.0, 122.0, 121.9, 119.8, 118.9, 118.9, 118.8, 117.5, 115.9, 113.6, 74.2, 52.1, 44.0, 30.7, 29.2.

### Example 1: Analysis of Multiple Changes in eIF2α Phosphorylation for N,N′-Squaramide Derivatives

Pathscan Phospho-eIF2α (Ser51) Sandwich ELISA Kit #7286 from Cell signaling was used. The K562 cell line was cultured in serum-free RPMI medium at 37° C. in the presence of 5% CO₂. Cells were treated with test compounds at a concentration of 10 μM. Six hours after compound treatment, the cells were lysed with a lysis buffer included in the kit to obtain 50 μg of protein by quantification. Afterwards, ELISA assay was performed according to the manufacturer's protocol to obtain absorbance. The absorbance for each obtained compound was standardized to a value of which the control (DMSO-treated) is set to 1.

TABLE 1

| No. | at 10 μM |
|---|---|
| 8 | 0.95 |
| 9 | 0.88 |
| 10 | 0.84 |
| 11 | 0.94 |
| 12 | 0.81 |
| 13 | 0.84 |
| 14 | 0.90 |
| 15 | 0.81 |
| 16 | 0.84 |
| 17 | 0.86 |
| 18 | 0.71 |
| 19 | 1.11 |
| 20 | 1.90 |
| 21 | 0.77 |
| 22 | 2.19 |
| 23 | 2.01 |
| 24 | 1.95 |
| 25 | 1.40 |
| 26 | 1.33 |
| 27 | 1.08 |
| 28 | 1.92 |
| 29 | 1.76 |
| 30 | 1.12 |
| 31 | 1.50 |
| 32 | 1.49 |
| 33 | 0.83 |
| 34 | 0.83 |
| 35 | 1.05 |
| 36 | 0.60 |
| 37 | 0.67 |
| 38 | 0.65 |
| 39 | 0.83 |
| 40 | 0.82 |
| 41 | 0.62 |
| 42 | 0.93 |
| 43 | 0.87 |
| 44 | 1.47 |
| 45 | 0.79 |
| 46 | 0.71 |
| 47 | 0.51 |
| 48 | 0.91 |
| 49 | 1.02 |
| 50 | 2.04 |
| 51 | 1.82 |
| 52 | 0.77 |
| 53 | 1.33 |
| 54 | 1.82 |
| 55 | 0.84 |
| 56 | 2.27 |
| 57 | 2.11 |
| 58 | 0.97 |
| 59 | 0.83 |
| 60 | 0.97 |
| 61 | 1.48 |
| 62 | 1.82 |
| 63 | 0.75 |
| 64 | 1.33 |
| 65 | 1.07 |
| 66 | 1.59 |
| 67 | 1.99 |
| 68 | 2.09 |
| 69 | 1.01 |
| 70 | 1.94 |
| 71 | 1.80 |
| 72 | 1.49 |
| 73 | 0.84 |
| 74 | 1.69 |
| 75 | 1.62 |
| 76 | 1.39 |
| 77 | 1.70 |
| 78 | 2.13 |
| 79 | 2.38 |
| 80 | 1.33 |
| 81 | 1.52 |
| 82 | 1.36 |
| 83 | 1.84 |
| 84 | 1.43 |
| 85 | 1.16 |
| 86 | 1.68 |

### Example 2: Analysis of Anti-Proliferative Activity of N,N′-Squaramide Derivatives for K562

CCK-8 assay (cell counting kit-8 assay, Dojindo, Kumamoto, Japan) was performed. The K562 cell line was cultured in RPMI medium supplemented with 10% FBS and 1% ABAM at 37° C. in the presence of 5% CO₂. Cells were treated with test compounds at concentrations of 0 μM, 1.25 μM, 2.5 μM, 5 μM, 10 μM, and 20 μM, respectively. After culture for 3 days, 10 μL of CCK-8 solution was treated and cultured for 4 hours. Subsequently, the absorbance was measured at 450 nm to obtain the IC₅₀ value of each compound.

TABLE 2

| | IC₅₀ (μM) |
|---|---|
| 19 | >20.00 |
| 20 | 16.09 |
| 23 | 3.96 |
| 24 | 5.94 |
| 25 | 12.25 |
| 26 | >20.00 |
| 28 | 6.80 |
| 29 | 4.09 |
| 30 | 5.09 |
| 31 | 5.69 |
| 35 | >20.00 |
| 49 | >20.00 |
| 70 | >20.00 |
| 71 | >20.00 |
| 72 | >20.00 |
| 83 | 4.00 |
| 84 | 10.07 |
| 85 | >20.00 |
| 86 | 14.23 |

### Example 3: Analysis of Selectivity Indices (SIs) for K562 and PBMC Cell Lines

The experimental values for the K562 cell line are identical to biological Example 2.

PBMC (peripheral blood monocyte cells isolated from normal donors) cell lines were dispensed in RPMI medium supplemented with 10% FBS and 1% ABAM at 37° C. in the presence of 5% CO₂, and then the test compounds were treated at concentrations of 0 μM, 1.25 μM, 2.5 μM, 5 μM, 10 μM, and 20 μM, respectively. After culture for 3 days, 10 μL of CCK-8 solution was treated and cultured for 4 hours. Subsequently, the absorbance at 450 nm was measured to obtain the IC₅₀ value of each compound.

The results of SI measurement in K562 cells compared to PBMC, which are normal cells, are as shown in Table 3.

TABLE 3

| | IC₅₀ | | |
|---|---|---|---|
| comp | K562 | PBMC | SI |
| 20 | 16.09 | 19.98 | 1.40 |
| 23 | 3.96 | 10.82 | 2.77 |
| 24 | 5.94 | 22.46 | 3.82 |
| 25 | 12.25 | 16.00 | 1.27 |
| 28 | 6.80 | 19.37 | 2.86 |
| 29 | 4.09 | 17.02 | 3.82 |
| 30 | 5.09 | 12.79 | 2.61 |
| 31 | 5.69 | 35.05 | 6.10 |
| 83 | 4.00 | 19.30 | 4.46 |
| 84 | 10.07 | 29.37 | 2.26 |
| 86 | 14.23 | 21.03 | 1.31 |

Having described the specific part of the present disclosure in detail above, for those skilled in the art, it is clear that the specific description is merely preferred example embodiments, and the scope of the present disclosure is not limited thereby. Accordingly, the substantial scope of the present disclosure will be defined by the appended claims and equivalents thereof.

1. A compound selected from a squaramide derivative compound represented by the following Chemical Formula 2 or Chemical Formula 3, a pharmaceutically acceptable salt thereof, a solvent thereof, or a stereoisomer thereof:

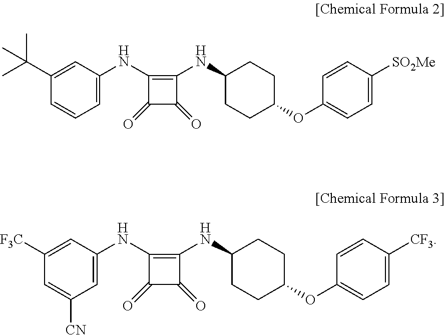 [SMILES: CC(C)(C)c1cccc(Nc2c(N[C@H]3CC[C@H](Oc4ccc(S(C)(=O)=O)cc4)CC3)c(=O)c2=O)c1 ; N#Cc1cc(Nc2c(N[C@H]3CC[C@H](Oc4ccc(C(F)(F)F)cc4)CC3)c(=O)c2=O)cc(C(F)(F)F)c1]

2. The compound selected from the squaramide derivative compound, pharmaceutically acceptable salt thereof, solvent thereof, or stereoisomer thereof of claim 1, wherein the squaramide derivative compound represented by the Chemical Formula 2 is 3-((3-(tert-butyl)phenyl)amino)-4-(((1,4-trans)-4-(4-(methylsulfonyl)phenoxy)cyclohexyl)amino)cyclobut-3-ene-1,2-dione.

3. The compound selected from the squaramide derivative compound, pharmaceutically acceptable salt thereof, solvent thereof, or stereoisomer thereof of claim 1, wherein the squaramide derivative compound represented by the Chemical Formula 3 is 3-((3,4-dioxo-2-(((1,4-trans)-4-(4-(trifluoromethyl)phenoxy)cyclohexyl)amino)cyclobut-1-en-1-yl)amino)-5-(trifluoromethyl)benzonitrile.

4. A method of preventing or treating cancer, comprising:
administering a pharmaceutical composition comprising a compound as an active ingredient to a subject, the compound selected from a squaramide derivative compound represented by the following Chemical Formula 2 or Chemical Formula 3, a pharmaceutically acceptable salt thereof, a solvent thereof, or a stereoisomer thereof:

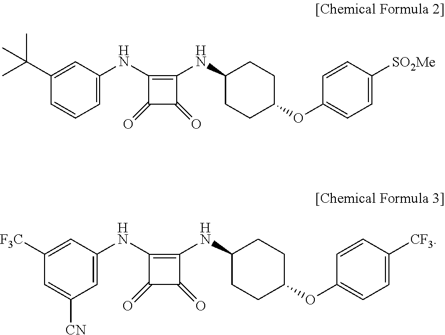 [SMILES: CC(C)(C)c1cccc(Nc2c(N[C@H]3CC[C@H](Oc4ccc(S(C)(=O)=O)cc4)CC3)c(=O)c2=O)c1 ; N#Cc1cc(Nc2c(N[C@H]3CC[C@H](Oc4ccc(C(F)(F)F)cc4)CC3)c(=O)c2=O)cc(C(F)(F)F)c1]

5. The method of claim 4, wherein the squaramide derivative compound represented by the Chemical Formula 2 is 3-((3-(tert-butyl)phenyl)amino)-4-(((1,4-trans)-4-(4-(methylsulfonyl)phenoxy)cyclohexyl)amino)cyclobut-3-ene-1,2-dione.

6. The method of claim 4, wherein the squaramide derivative compound represented by the Chemical Formula 3 is 3-((3,4-dioxo-2-(((1,4-trans)-4-(4-(trifluoromethyl)phenoxy)cyclohexyl)amino)cyclobut-1-en-1-yl)amino)-5-(trifluoromethyl)benzonitrile.

7. The method of claim 4, wherein the compound promotes phosphorylation of eukaryotic translation initiation factor 2α (eIF2α).

8. The method of claim 4, wherein the cancer is blood cancer, breast cancer, sarcoma cancer, or brain cancer.

9. A method of inhibiting cancer cell metabolism, comprising
administering a pharmaceutical composition comprising a compound as an active ingredient to a subject, the compound selected from a squaramide derivative compound represented by the following Chemical Formula 2 or Chemical Formula 3, a pharmaceutically acceptable salt thereof, a solvent thereof, or a stereoisomer thereof:

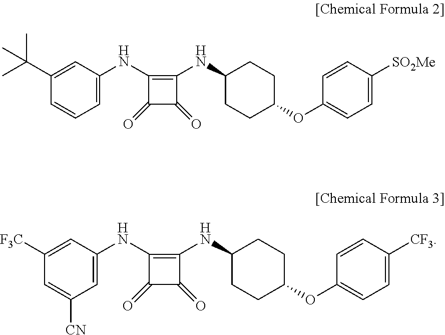 [SMILES: CC(C)(C)c1cccc(Nc2c(N[C@H]3CC[C@H](Oc4ccc(S(C)(=O)=O)cc4)CC3)c(=O)c2=O)c1 ; N#Cc1cc(Nc2c(N[C@H]3CC[C@H](Oc4ccc(C(F)(F)F)cc4)CC3)c(=O)c2=O)cc(C(F)(F)F)c1]

10. The method of claim 9, wherein the squaramide derivative compound represented by the Chemical Formula 2 is 3-((3-(tert-butyl)phenyl)amino)-4-(((1,4-trans)-4-(4-(methylsulfonyl)phenoxy)cyclohexyl)amino)cyclobut-3-ene-1,2-dione.

11. The method of claim 9, wherein the squaramide derivative compound represented by the Chemical Formula 3 is 3-((3,4-dioxo-2-(((1,4-trans)-4-(4-(trifluoromethyl)phenoxy)cyclohexyl)amino)cyclobut-1-en-1-yl)amino)-5-(trifluoromethyl)benzonitrile.

12. The method 9, wherein the compound promotes phosphorylation of eukaryotic translation initiation factor 2α (eIF2α) to block metabolism of cancer cells.
